(19) United States
(12) Patent Application Publication
(10) Pub. No.: US 2026/0242808 A1
(43) Pub. Date: Aug. 20, 2026

(54) TRANS-COMPLEMENTARY VECTOR SYSTEM FOR HIGHLY EFFICIENT HETEROLOGOUS GENE EXPRESSION IN PLANTS

(71) Applicant: SWIFTPHAMA BV, Gent (BE)

(72) Inventor: Jeroen HOFENK, Eindhoven (NL)

(21) Appl. No.: 19/471,921

(22) PCT Filed: Apr. 3, 2024
(86) PCT No.: PCT/EP2024/059083

§ 371 Date: Oct. 2, 2025

(30) Foreign Application Priority Data
Apr. 3, 2023 (EP) 23166367.5

(51) Int. Cl. C12N 15/82

(52) U.S. Cl. CPC C12N 15/8205; C12N 15/8203

(57) ABSTRACT

The present invention relates to a trans-complementary system for heterologous expression of genes in plants and comprises a producer vector and a helper vector whereas I) said producer vector is incapable for at least one function needed to cause systemic infection in plants e.g., either by replication, cell-to-cell and/or long distance movement, and II) the helper vector completes the missing function in tandem. Certain producer and helper vectors in one trans-complementary system are derived from the same virus, or certain producer and helper vectors in one trans-complementary system are derived from different plant viruses. The present invention also provides methods of use, e.g., to improved yield of the heterologous proteins expressed notably in crop plants, and the ease of use of said system.

The present invention relates to a trans-complementary system for heterologous expression of genes in plants and comprises a producer vector and a helper vector whereas 1) said producer vector is incapable for at least one function needed to cause systemic infection in plants e.g., either by replication, cell-to-cell and/or long distance movement, and II) the helper vector completes the missing function in tandem. Certain producer and helper vectors in one trans-complementary system are derived from the same virus, or certain producer and helper vectors in one trans-complementary system are derived from different plant viruses. The present invention also provides methods of use, e.g., to improved yield of the heterologous proteins expressed notably in crop plants, and the ease of use of said system.

### TECHNICAL FIELD AND INDUSTRIAL APPLICABILITY OF THE INVENTION

This invention comprises expression vectors from viruses selected from the group of Tobravirus and particularly viral derived sequences, methods for modifying such vectors, boosting gene expression in plants and uses of the same.

### STATEMENT REGARDING FEDERALLY SPONSORED RESEARCH

This invention was not made with any Government support and the government has no rights in this invention.

### BACKGROUND OF THE INVENTION

Over the last three decades, plants have been increasingly used as an expression host for the heterologous expression of various recombinant proteins. As such, this heterologous expression can be accomplished either by integrating the gene of interest (GOI) into the plant genome, thereby creating a stable transformed or “transgenic” plant which stably expresses the target protein, or by introduction of the GOI into a plant vector that can be introduced into the plant host and maintained transiently in said plant cells, thereby preventing the GOI to be replicated.

According to the current opinion, the technology of transient gene expression systems in plant systems has considerable advantages over stable expression, namely, (I) it does not require regeneration of the transformed cell; (II) has no effect on the stability of host genome; and (III) is independent of the position effects of the T-DNA integration sites. Transient expression is firstly made possible by agroinfiltration or agroinfection (Grimsley et al. 1985). In agroinfection, a plant functional promotor and RNA- or DNA-based virus cDNA are transferred as T-DNA from Agrobacterium into plant cells. The T-DNA is transcribed in planta, to generate biologically active viral RNAs that can initiate self-replication.

Plant virus-based vectors allow for this rapid, transient expression of target proteins in whole plants (Pogue et al. 2002, Scholthof et al. 2002, Yusibov et al. 1999, Grimsley et al. 1986, Turpen et al. 1993) and numerous publications and patents in the field describe systems based on DNA and RNA viral vectors (Kumagai et al. 1994, Mor et al. 2003, U.S. Pat. Nos. 5,316,931, 5,589,367, 58,866,785, 5,491,076, 5,981,236, EP1706497, U.S. Pat. Nos. 8,951,791, 8,936,937, WO1998054342, WO02088369, WO2009087391).

The existing viral vector systems are usually restricted to a narrow host range in terms of their best performance and even the expression level of such vectors in their most favorable host is far below the upper biological limit of plant expression systems.

Recently, an agroinfection-compatible TMV expression vector was constructed by extensive modifications such as multiple mutations, destruction of cryptic introns, and insertions of multiple plant-gene introns into the TMV cDNA sequences in a binary vector (Marillonnet et al. 2005) named “MagnICON” (Gleba et al. 2005, Marillonnet et al. 2004, EP1706497). Although these modifications improved the efficiency by which TMV vectors can be introduced into plants, the MagnICON vector approach is not easily adapted to a high throughput workflow.

The magnlCON system has been used to produce a wide variety of proteins in plants, including plague antigens (Santi et al., 2006), hepatitis B virus core antigen (HBcAg) virus-like particles (VLPs) (Huang et al., 2006) and Norwalk virus VLPs (Santi et al., 2008). The production of antibodies was, for a time, more problematic. Soon after the development of the MagnICON system, it became clear that viral competition would make it difficult to co-express multiple replicons in the same cells, which would make it impossible to produce oligomultimeric proteins like immunoglobulin G (IgG) (Gleba et al., 2005).

The solution to this problem was to express one chain of the IgG using MagnICON, and the other chain using a different noncompeting viral vector system, based on potato virus X (PVX). This allowed the production of assembled IgG in N. benthamiana (Giritch et al., 2006). Since then, IgG have been produced using this noncompeting viral vectors strategy in an industrial setting as candidate vaccines for hon-Hodgkin's lymphoma (Bendandi et al., 2010).

Subsequent to the first reports on MagnICON, a single-module TMV-based overexpression vector was developed. The TRBO system (U.S. Pat. No. 8,936,937) is based on a 35S promoter-led replicon based on the TMV genome from which the CP gene, under the control of the native subgenomic promoter present within the MP gene, is replaced with a multiple cloning site into which the GOI is inserted (Lindbo, 2007). Like MagnICON, TRBO replicons cannot move systemically, but they can move cell to cell within an infected leaf, so agroinfiltration is crucial to initiating an infection of the entire plant. The author demonstrated that the co-expression of an exogenous suppressor of gene silencing had no effect on transgene expression. However, this was not the case for versions of the expression vector that still contained the CP gene, which led the author to suggest that this sequence may be an inducer of gene silencing.

Moreover, it has since been determined that suppressor of silencing activity is in fact already present on the replicase protein of TMV (Wang et al., 2012). In addition to this, the initiation of primary replicons in agroinfected cells seemed to be just as efficient as the improved MagnICON vectors, but without the need to insert numerous exogenous sequences such as introns into the tobamovirus genes. The author suggests that this may be due to the fact that TRBO is based on the U1 strain of TMV, while MagnICON is based on turnip vein clearing virus (TVCV) and the crucifer-infecting strain of TMV (cr-TMV). It therefore appears that a TRBO vector has all of the advantages of a MagnICON vector, being smaller and simpler in design.

Other transient expression systems are based on a variety of single- or double-stranded plant viruses such as potexvirus (Baulcome et al. 1995, Santa Cruz et al. 1996, Smolenska et al. 1998, O'Brien et al. 2000, Lico et al. 2006, Mardanova et al. 2009, Larsen and Curtis, 2012), tobravirus (Constantin et al. 2004, Deng et al. 2013, MacFarlane, 2010, Yang et al. 2013, MacFarlane and Popovich, 2000, Swanson et al. 2002), geminivirus (Mor et al. 2003, Zhang and Mason, 2006, Dugdale et al. 2013, Huang et al. 2010, Sainsbury and Lomonossoff, 2008), and comovirus (Sainsbury et al. 2010, Porta et al. 1994, Usha et al. 1993, Gopinath et al. 2000, Sainsbury and Lomonossoff, 2014, Peyret and Lomonossoff, 2013).

Thus, by observation from prior there remains a high unmet need for developing improved systems for the heterologous expression of transgenes in plants that: I) contain convenient cloning sites for GOI's; II) can be used to infect plants in an easy and cost-effective manner, III) lead to efficient systemic infection of inoculated plants to obtain high yields of recombinant product, IV) minimize the risk of environmental contamination (e.g., viruses that may infect non-target plants).

In addition, as interest in proteomics, biochemistry and protein structure increases there is an increasing need for efficient, easy-to-use recombinant protein expression systems. Improving transient expression vectors so they are easier to use, more cost-effective and produce higher levels of target proteins in competitive large-scale manufacturing will be of great use.

### SUMMARY OF THE INVENTION

The present invention encompasses the recognition that there is a need to develop expression systems for plants that present only a minimal risk of environmental contamination and provides methods and reagents for expression of polynucleotides in plants with a reduced risk of widespread contamination.

Some particular definitions and embodiments of the present invention will now be described in more detail. Various objects and advantages of this invention will become apparent to those skilled in the art from the following detailed description of the preferred embodiments, when read in light of the accompanying drawings and claims. This application refers to various patents, patent applications, and publications. The contents of all of these are incorporated herein by reference. In addition, the following publications are incorporated herein by reference: Current Protocols in Molecular Biology, Current Protocols in Immunology, Current Protocols in Protein Science, and Current Protocols in Cell Biology, all John Wiley & Sons, N.Y., volume editions as of 2020.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

Notwithstanding the exemplary embodiments described hereinbelow, is the present invention only limited by the attached claims. The attached claims are hereby explicitly incorporated in this detailed description, in which each claim, and each combination of claims as allowed for by the dependency structure defined by the claims, forms a separate embodiment of the present invention.

Reference throughout this specification to “one embodiment” or “an embodiment” or “embodiments” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim.

Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

The non-essential improvement and adjustment made by the method disclosed by the invention should still be within the protection scope of the invention. Meanwhile, raw materials being used are not always described in detail. The raw materials may be commercially available products. The process steps or preparation methods that are not always described in detail are process steps or preparation methods which are known by those skilled in the art.

The raw materials used to obtain the present invention are commercially available products; the process steps or the preparation method which are also not always described in detail, are process steps or preparation methods which are all known by those skilled in the art.

As used herein the term “about” refers to ±10%.

The term “consisting of means “including and limited to”.

The term “consisting essentially of” means that the composition, process or structure may contain additional components, steps and/or parts, but only if the additional components, steps and/or parts meet the basic and novel properties of the claimed composition and therefor do not materially alter the basic and novel characteristics of the claimed composition and/or method or structure.

As used herein, the singular forms “a”, “an” and “the” include plural references unless the context clearly indicates otherwise. For example, the term “a compound” or “at least one compound” can encompass a variety of compounds, including mixtures thereof which can be used in foods, cosmetics, pharmaceuticals, industrial products, medical products, laboratory culture growth media, and many other applications.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

In the description provided herein, numerous specific details are set forth. However, it is understood that embodiments of the invention may be practiced without these specific details. In other instances, well-known methods, structures and techniques have not been shown in detail in order not to obscure an understanding of this description.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof.

In the present described invention, various specific details are presented. Embodiments of the present invention can be carried out without these specific details. Furthermore, well-known features, elements and/or steps are not necessarily described in detail for the sake of clarity and conciseness of the present disclosure.

In one aspect, there are provided modified Tobacco Rattle Virus (TRV) expression vectors and novel methods for modifying said TRV vectors. The methods provide modifications that improve both the options for cloning genes into TRV and the ease and efficiency of infecting plants with TRV vectors.

The present invention comprises a system for replicating or for replicating and expressing a sequence of interest in a plant host, comprising:
I) a producer vector enabling in trans cell-to-cell movement of the trans-complementary viral replicon;
II) a helper vector, wherein said helper vector is i) capable of systemic movement in said plant host both in the presence of said producer vector, and ii) capable of expressing a polynucleotide of interest in said plant host by the help of one or more proteins necessary for systemic movement, either by replication, cell-to-cell and/or long distance movement of with the help of said producervector; and
III) an integrase vector which enables the in planta assembly of component i) and ii), being the producer and helper vector, thereby enabling the resulting components to be recombined inside the plant cell and leading to the creation of a vector sequence within a fully functional infective replicon

Wherein said producer vector is capable of replicating or replicating and expressing said GOI in said plant host, but unable to move systemically in said plant host in the absence of said one or more proteins expressed by said helper vector, as further defined by claim 1.

The present invention further provides a process of replicating or a process of replicating and expressing a GOI in a plant host, comprising providing cells of said plant host with said producer vector and said helper vector, as defined by the claims. In a particular embodiment the present invention further provides a process of replicating or a process of replicating and expressing a GOI in a plant host, comprising providing cells of said plant host with said producer vector, said helper vector, and said integrase vector, as defined by the claims. The invention may be used for replicating a GOI in a plant host or for replicating and expressing said GOI, e.g., for the production of said GOI in said plant host. Furthermore, the present invention provides a process of polypeptide production in plant hosts, or in plant host cells.

In certain embodiments of the present invention the helper vector is dysfunctional to carry out replication. For example, the producer vector may include an RNA polymerase coding sequence (e.g., a replicase coding sequence) and a movement protein coding sequence so that said producer vector is capable to carry out cell-to-cell movement, but may lack a CP coding sequence so that said producer vector is not capable to carry out long-distance systemic movement.

Therefore, the present invention reduces or eliminates the risk of spreading to non-target plants accompanied with (recombinant) vectors, thus thereby significantly improving the environmental safety of heterologous gene expression in plant hosts and furthermore allowing more flexibility in the cultivation of recipient plant hosts.

The present invention further provides vector systems that can be used as components for other purposes. For instance, present invention offers those who are skilled in the art, e.g., a researcher, the flexibility to design a plant expression system with qualities of more than one plant virus. For example, in specific embodiments the producer vector desirably has the GOI positioned such that its expression is controlled by the CP promotor. Therefore, in many cases it is desirable to base the producer vector on a viral system with a strong CP promotor.

However, viruses with strong CP promotors may sometimes have limited plant host specificity because they may be unable to infect, replicate within and/or enable cell-to-cell or long distance movement within certain plant hosts. Therefore, it may be desirable to base the helper vector on a viral system with a broad plant host specificity, thereby enabling exploiting a host plants that would otherwise ordinarily be inaccessible to that viral system.

In addition, the inventor has found that despite the increase of protein expression of a particular system in the embodiments of the present invention, plant hosts transformed with a trans-complementary TRV vector of said present invention, looked healthier, i.e., showed less necrosis, compared to plants transformed with a known TRV vector (vector pYL192 (TRV1), addgene plasmid N° 1489968) (FIG. 19B). Plant health is essential in the downstream purification of GOIs as tannins that are released as a result of necrosis, can interfere with protein purification (Chatterjee et al. 2012), thereby contributing to an increase in operational expenses (COGs) which in turn results in higher costs of goods sold (COGS).

In another aspect, the cloning options in the present invention were improved by the development of a simple, novel and inexpensive method for cloning DNA inserts into the agroinfection-compatible trans-complementary vector systems. The cloning method allows for the efficient, site-directional of PCR products, without the need for restriction enzyme digestion of PCR products. The cloning method does not rely on expensive topoisomerase or recombinase enzymes uses commonly available enzymes known by those skilled in the art. It therefore enables the use of said vectors in high-throughput experiments.

In one particular aspect, the present invention provides an improvement to transient expression technology known in the prior art. In particular, the present invention provides a system for improving trans-complementary transient expression vectors for the very high level of heterologous protein expression in plant hosts, where said vectors are efficiently delivered to plant cells by agroinfection.

### Definitions

For the purposes of the present invention, the following terms have their meaning as understood by those skilled in the art.

“GENE”: In general, a gene includes gene regulatory coding sequences such as promotors, enhancers, and terminators and/or intron sequences, in addition to coding sequences or open reading frames (ORFs). The definition of gene can included nucleic acids that do not encode proteins but rather provide templates for transcription of functional RNA molecules.

The inventor notes for the purpose of clarity that, as used in the present invention, the term “gene” generally refers to a nucleic acid that includes a portion encoding a protein; the term may also encompass regulatory coding sequences such as promotors, enhancers, terminators, etc. This definition is not intended to exclude application of the definition “gene” non-protein coding expression units but rather to clarify that, in most cases, the term as used in this present invention refers to a protein coding nucleic acid. “GENE PRODUCT” or “EXPRESSION PRODUCT”: in general an RNA transcribed from the gene or a polypeptide encoded by an RNA transcribed from the gene. Expression of a gene or a polynucleotide refers to I) transcription of RNA from the gene or polynucleotide, or II) translation of RNA transcribed from the gene or polynucleotide, or both I and II.

“ISOLATED”: as referred herein, a compound that is separated from the components from which it is normally associated or produced or prepared by a process that involves the hand of man.

“NATIVE” or “NATURAL”: means “naturally-occurring” and as used herein, refers to things or components that occur in their relevant form in nature. By contrast “not naturally-occurring” refers to things or components whose form results from the hand of man.

“OPERABLY LINKED” or “OPERABLE LINKER”: as referred herein, means the relationship between two individual nucleic acids or coding sequences, wherein the expression of one of the coding sequences is controlled by, regulated by, modulated by, etc. the other coding sequence. It is noted that a single coding sequence can be operably linked to multiple other coding sequences. For example, a single promotor coding sequence can direct transcription of multiple RNAs.

“GOI”; as referred herein, means gene-of-interest and refers to any target coding sequence to be expressed in a plant host. In a variety of embodiments of the present invention, the GOI may be a coding sequence of a protein, but may also be a coding sequence that provides a template for transcription of a structural RNA or an active RNA such as a ribozyme, interfering RNA, and the like. Often so, the GOI will be a gene that is not expressed in nature in the relevant type of plant cell (e.g., heterologous expression), or is not expressed at the level that the GOI is expressed when expression is achieved by intervention of the hand of man. Furthermore, the GOI is one that is not naturally associated with the vector coding sequences of the present invention. The abbreviation “GOI” is used interchangeably with “nucleic acid” or “nucleic acid molecule” and are referred to herein as a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after the assembly of the polymer. The sequence of nucleotides may be interrupted by non-nucleotide components. A polynucleotide may be further modified after polymerization, such as by conjugation with a labelling component.

“EXOGENOUS RNA SEGMENT”: as referred herein, describes a segment of RNA to be inserted into the virus RNA to be modified, the source of the exogenous RNA segment being different from the RNA virus itself. The source may be another virus, a living organism such as a plant, animal, bacteria, or fungus. The exogenous RNA may be chemically synthesized RNA or it may be a combination of the foregoing. The exogenous RNA segment may provide any function that is appropriate and know to be provided by an RNA segment. Such functions include, but are not limited to, a coding function in which the RNA acts as a messenger RNA encoding a sequence which, translated by the host cell, results in synthesis of a polypeptide or protein having useful or desired properties. The RNA segment may also be structural, as for example in ribosomal RNA, it may be regulatory, as for example with small nuclear RNAs or anti-sense RNA, or it may be catalytic. An exogenous RNA segment can be a complete or partial coding sequence.

“SELF-REPLICATE” or “SELF-REPLICATION”: as referred herein, means a vector having the ability to copy itself, and carrying sufficient information in its own genetic elements that it does not rely on other genetic elements for its replication inside a plant host cell.

“VECTOR”: refers to a nucleic acid molecule capable of transporting another nucleic acid molecule to which it has been linked and can include a plasmid, cosmid or viral vector.

“PLASMID”; refers to autonomous extrachromosomal genetic elements found in many kinds of bacteria. They consist of closed circular DNA. Some can become integrated into the host chromosome and replicate with it. Some viruses that infect prokaryotes have properties like those of plasmids, and in particular to integrate into the host cell chromosome. Preferred vectors in the present invention are maintained extra-chromosomally.

“COMPONENT”; referred to herein, is intended to include both protein coding sequences and non-coding sequences such as cis-acting sequences (e.g., promoters, origin of assembly, portions corresponding to UTRs in mRNA).

“PHENOTYPIC TRAIT”; as referred to herein, is an observable, measurable, or detectable property resulting from the expression or suppression of a gene or genes. “Phenotype” includes both easily observable traits and biochemical processes.

“PROMOTOR”; as referred to herein, is the 5′-flanking, non-coding sequence substantially adjacent a coding sequence which is involved in the initiation of transcription of the coding sequence.

### DETAILED DESCRIPTION OF PREFERRED EMBODIMENTS OF THE PRESENT INVENTION

### Inventive Vectors

As duly noted hereabove, this present invention discloses systems for the heterologous expression of GOIs in plant hosts. These systems may include one or more viral components and as such, a plethora of plant viruses are known to infect a variety of plant species. These can be deployed for heterologous expression according to preferred embodiments of the present invention. FIG. 1. Represents a schematic representation of virus families infecting plants. Additional information can be found, for example in Virus Taxonomy, Ninth Report of the International Committee on Taxonomy of Viruses (Andrew MQ King, Elliot Lefkowitz et al., 2011), the entire contents of which are incorporated herein by reference.

A virus is a set of one or more nucleic acid template molecules, normally encased in a protective coat or coats of protein or lipoprotein, that is able to organize its own replication only within suitable host cells. Within such cells, virus replication is; I) dependent on the host's protein-synthesizing machinery, II) organized from pools of the required materials rather than by binary fission, III) located at sites that are not separated from the host cell contents by a lipo-protein bilayer membrane, and IV) continually giving rise to variants through several kinds of change in the viral nucleic acid.

Plants (crops, medicinal or ornamental), can be infected by viruses. It all may start with an insect bite, a nematode, physical damage to the cell, or by micro-organisms such as a bacteria, fungi, or archaea.

The virus only has to reach a single cell to initiate infection. However, as viruses cannot do anything by themselves, they need to hijack the infected cell's mechanisms to produce copies of themselves. So first, plant viruses need to cross the cell wall, in addition to protective layers consisting of waxes and pectins.

Some types of enveloped virus fuse directly to the cell's outer (plasma) membrane, whereas the majority are engulfed whole by endocytosis (mechanical breach) or similar processes and then fuse their envelope with the membrane of the engulfing internal organelle (e.g., an endosome) to gain access to the interior of the cell. Eventually, progeny viruses are released to neighboring cells and this cycle is repeated continuously.

Soon the virus is able to reach the vascular system in the plant (similarly to the circulatory system in animals) and can spread long distances from the initial spot of infection, infecting everything from roots to young leaves. In the laboratory, viruses are typically administered to plant cells simply by bringing the virus into contact (rubbing or forced entry) with the plant.

The structure and replication of viruses have the following features: I) the infectious nucleic acid may be DNA or RNA (but never both) and be single- or double-stranded. If the nucleic acid is single-stranded it may be of positive or negative sense. (Positive sense has the sequence that would be used as an mRNA for translation to give a virus-coded protein), II) the mature virus particle may contain polynucleotides other than the genomic nucleic acid, III) where the genetic material consists of more than one nucleic acid molecule, each may be housed in a separate particle or all may be located in one particle, IV) the genomes of viruses vary widely in size, encoding between 1 and about 250 proteins. Plant viral genomes are at the small end of this range, mostly encoding between 1 and 12 proteins. The plant virus-coded proteins may have functions in virus replication, in virus movement from cell to cell or long-distance, in virus structure, and in transmission by invertebrates or fungi. In general, many viruses contain genes encoding one or more proteins that participate in the replication process (referred to herein as replication proteins or replicates proteins). For example, many RNA plant viruses encode an RNA polymerase. V) viruses undergo genetic change. Point mutations occur with high frequency as a result of nucleotide changes brought about by errors in the copying process during genome replication. Other kinds of genetic change may be due to recombination, reassortment of genome pieces, loss of genetic material, or acquisition of nucleotide sequences from unrelated viruses or the host genome, VI) enzymes specified by the viral genome may be present in the virus particle. Most of these enzymes are concerned with nucleic acid synthesis, VII) replication of many viruses takes place in distinctive virus-induced structures in the plant cell, VIII) some viruses share with certain non-viral nucleic acid molecules the property of integration into host-cell genomes and translocation from one integration site to another, IX) a few viruses require the presence of another virus for their replication.

In general, for a virus to spread from cell-to-cell it requires a functional movement and coat protein, while systemic (long distance) spread requires a functional coat protein, and more generally, also a functional movement protein. Additionally, viruses may also contain other components that are either required for local or systemic spread or to facilitate such spread. These “cis-acting” regulatory components may be either coding or non-coding components. For instance, they may correspond to portions of a 3′UTR of a viral transcript.

Any virus that infects plants may be used to prepare a viral vector or vector system in accordance with the present invention. Particularly preferred viruses have a positive (+)-stranded genome, either single-stranded (ss) or double-stranded (ds). The use of techniques and reagents forthe manipulation of genetic material present in such viruses are well known by those skilled in the art. Generally, a DNA copy of the viral genome is prepared and cloned into a bacterial vector.

Certain ssDNA viruses, preferably Mastreviruses within the Geminiviridae, are also of particular interest. It will be appreciated even more so, that in general the vectors and viral genomes of the present invention may exist in either RNA or DNA form. Additionally, were reference is to be made to a feature such as a genome or part thereof of an RNA virus, which is present within a DNA vector, it is to be understood by those skilled in the art that the feature is present as the DNA copy of the RNA form.

As described in the preferred embodiments of the present invention, a variety of different types of viruses may be used either independently or in combination with each other. Preferred viruses of the Virgaviridae family include Tobacco Rattle Virus, Pea Early-Browning Virus, and Pepper Ringspot Virus, but more preferably Tobacco Rattle Virus being used. Preferred viruses of the Comovirinae sub-family include Cowpea Mosaic Virus, Andean Potato Mottle Virus, Bean Pod Mottle Virus, Bean Rugose Mosaic Virus, Broad Bean Stain Virus, Broad Bean True Mosaic Virus, Cowpea Severe Mosaic Virus, Glycine Mosaic Virus, Pea Green Mottle Virus, Pea Mild Mosaic Virus, Quail Pea Mosaic Virus, Radish Mosaic Virus, Red Clover Mottle Virus, Squash Mosaic Virus, Ullucus Virus C, but more preferably Cowpea Mosaic Virus being used. Preferred viruses of the Potyviridae family include Tobacco Etch virus, Barley Yellow Mosaic Virus, Bellflower Veinal Mottle Virus, Blackberry virus Y, Maclura Mosaic Virus, Potato Virus Y, Rose Yellow Mosaic Virus, Ryegrass Mosaic Virus, Sweet Potato Mild Mottle Virus, Triticum Mosaic Virus, Wheat Streak Mosaic Virus, Agropyron Mosaic Virus, Algerian Watermelon Mosaic Virus, Alpinia Mosaic Virus, Alstroemeria Mosaic Virus, Alternanthera Mild Mosaic Virus, Amaranthus Leaf Mottle Virus, Amazon Lily Mosaic Virus, Angelica Virus Y, Apium Virus Y, Araujia Mosaic Virus, Arracacha Mottle Virus, Artichoke Latent Virus, Asparagus Virus 1, Banana Bract Mosaic Virus, Barbacena Virus Y, Barley Mild Mosaic Virus, Basella Rugose Mosaic Virus, Bean Common Mosaic Necrosis Virus, Bean Common Mosaic Virus, Bean Yellow Mosaic Virus, Beet Mosaic Virus, Bidens Mosaic Virus, Bidens Mottle Virus, Blue Squill Virus A, Broad-Leafed Dock Virus A, Brome Streak Mosaic Virus, Brugmansia Mosaic Virus, Brugmansia Suaveolens Mottle Virus, Butterfly Flower Mosaic Virus, Caladenia Virus A, Calanthe Mild Mosaic Virus, Calla Lily Latent Virus, Callistephus Mottle Virus, Canna Yellow Streak Virus, Cardamom Mosaic Virus, Carnation Vein Mottle Virus, Carrot Thin Leaf Virus, Carrot Virus Y, Cassava Brown Streak Virus, Catharanthus Mosaic Virus, Celery Mosaic Virus, Ceratobium Mosaic Virus, Chilli Ringspot Virus, Chilli Veinal Mottle Virus, Chinese Artichoke Mosaic Virus, Chinese Yam Necrotic Mosaic Virus, Clitoria Virus Y, Clover Yellow Vein Virus, Coccinia Mottle Virus, Cocksfoot Streak Virus, Colombian Datura Virus, Commelina Mosaic Virus, Cowpea Aphid-Borne Mosaic Virus, Cucumber Vein Yellowing Virus, Cypripedium Virus Y, Cyrtanthus Elatus Virus A, Daphne Mosaic Virus, Daphne Virus Y, Dasheen Mosaic Virus, Datura Shoestring Virus, Diuris Virus Y, Donkey Orchid Virus A, East Asian Passiflora Virus, Endive Necrotic Mosaic Virus, Euphorbia Ringspot Virus, Freesia Mosaic Virus, Fritillary Virus Y, Gloriosa Stripe Mosaic Virus, Habenaria Mosaic Virus, Hardenbergia Mosaic Virus, Henbane Mosaic Virus, Hibbertia Virus Y, Hippeastrum Mosaic Virus, Hordeum Mosaic Virus, Hyacinth Mosaic Virus, Impatiens Flower Break Virus, Iris Fulva Mosaic Virus, Iris Mild Mosaic Virus, Iris Severe Mosaic Virus, Japanese Yam Mosaic Virus, Jasmine Virus T, Johnsongrass Mosaic Virus, Kalanchoe Mosaic Virus, Keunjorong Mosaic Virus, Konjac Mosaic Virus, Leek Yellow Stripe Virus, Lettuce Italian Necrotic Virus, Lettuce Mosaic Virus, Lily Mottle Virus, Lupinus Mosaic Virus, Lycoris Mild Mottle Virus, Maize Dwarf Mosaic Virus, Malva Vein Clearing Virus, Meadow Saffron Breaking Virus, Moroccan Watermelon Mosaic Virus, Narcissus Degeneration Virus, Narcissus Late Season Yellows Virus, Narcissus Latent Virus, Narcissus Yellow Stripe Virus, Nerine Yellow Stripe Virus, Nothoscordum Mosaic Virus, Oat Mosaic Virus, Oat Necrotic Mottle Virus, Onion Yellow Dwarf Virus, Ornithogalum Mosaic Virus, Ornithogalum Virus 2, Ornithogalum Virus 3, Panax Virus Y, Papaya Leaf Distortion Mosaic Virus, Papaya Ringspot Virus, Parsnip Mosaic Virus, Passiflora Chlorosis Virus, Passion Fruit Woodiness Virus, Pea Seed-Borne Mosaic Virus, Peanut Mottle Virus, Pecan Mosaic-Associated Virus, Pennisetum Mosaic Virus, Pepper Mottle Virus, Pepper Severe Mosaic Virus, Pepper Veinal Mottle Virus, Pepper Yellow Mosaic Virus, Peru Tomato Mosaic Virus, Pfaffia Mosaic Virus, Pleione Virus Y, Plum Pox Virus, Pokeweed Mosaic Virus, Potato Virus A, Potato Virus V, Ranunculus Leaf Distortion Virus, Ranunculus Mild Mosaic Virus, Ranunculus Mosaic Virus, Rhopalanthe Virus Y, Rice Necrosis Mosaic Virus, Sarcochilus Virus Y, Scallion Mosaic Virus, Shallot Yellow Stripe Virus, Sorghum Mosaic Virus, Soybean Mosaic Virus, Spartina Mottle Virus, Spiranthes Mosaic Virus 3, Squash Vein Yellowing Virus, Sugarcane Mosaic Virus, Sugarcane Streak Mosaic Virus, Sunflower Chlorotic Mottle Virus, Sunflower Mosaic Virus, Sunflower Ring Blotch Virus, Sweet Potato Feathery Mottle Virus, Sweet Potato Latent Virus, Sweet Potato Mild Speckling Virus, Sweet Potato Virus 2, Sweet, Potato Virus C, Sweet Potato Virus G, Tall Oatgrass Mosaic Virus, Tamarillo Leaf Malformation Virus, Telfairia Mosaic Virus, Telosma Mosaic Virus, Thunberg Fritillary Mosaic Virus, Tobacco Mosqueado Virus, Tobacco Vein Banding Mosaic Virus, Tobacco Vein Mottling Virus, Tomato Mild Mottle Virus, Tomato Necrotic Stunt Virus, Tradescantia Mild Mosaic Virus, Tuberose Mild Mosaic Virus, Tuberose Mild Mottle Virus, Tulip Breaking Virus, Tulip Mosaic Virus, Turnip Mosaic Virus, Twisted-Stalk Chlorotic Streak Virus, Ugandan Cassava Brown Streak Virus, Vallota Mosaic Virus, Vanilla Distortion Mosaic Virus, Verbena Virus Y, Watermelon Leaf Mottle Virus, Watermelon Mosaic Virus, Wheat Eqlid Mosaic Virus, Wheat Spindle Streak Mosaic Virus, Wheat Yellow Mosaic Virus, Wild Onion Symptomless Virus, Wild Potato Mosaic Virus, Wild Tomato Mosaic Virus, Wisteria Vein Mosaic Virus, Yam Chlorotic Mosaic Virus, Yam Mild Mosaic Virus, Yam Mosaic Virus, Yambean Mosaic Virus, Yellow Oat Grass Mosaic Virus, Zantedeschia Mild Mosaic Virus, Zea Mosaic Virus, Zucchini Shoestring Virus, Zucchini Tigre Mosaic Virus, Zucchini Yellow Fleck Virus, Zucchini Yellow Mosaic Virus, but more preferably Tobacco Etch Virus being used.

To generate viral vectors to be used in accordance with the present invention, a variety of elements of the aforementioned preferred plant viruses are genetically engineered in accordance with techniques known by those skilled in the art (examples are to be found in Molecular Cloning—A laboratory manual, 4^{th} edition by Michael R. Green and Joseph Sambrook, 2012, Plant Virology Protocols: From Virus Isolation to Transgenic Resistance, Methods in Molecular Biology, volume 81 by Gary Foster, etc.). According to the present invention, at least two vector are employed, one or both of which are depending on each other in order to support systemic functioning and heterologous expression of one or more GOIs, thus recognizing that viral components used in the present invention can complement each other in trans to facilitate being functional by trans-complementation.

In a particular embodiment of the present invention, a producer vector is prepared which includes a GOI that is placed under control of regulatory coding sequences that responsible for direct expression in a plant host. In preferred embodiments, a GOI is placed under the control of a viral promoter, for example by insertion of coding sequences containing a CP subgenomic RNA promotor. For example, where it is desired the natural viral CP gene may be replaced with a GOI.

The producer vector may lack one or more components that are essential for systemic movement. For example, in preferred embodiments of the present invention the producer vector does not contain coding sequences that are sufficient for expression of a functional CP gene, but may include a coding sequence that enables cell-to-cell-movement. The producer vector may contain one or more coding sequences as an origin of assembly, which may be required in cis to enable spread of the virus when present in cis. For example, the producer vector in certain embodiments of the present invention may contain an origin of assembly that is needed for- or enables activity of a CP coding sequence, either from the same type of virus as said producer virus or from another virus chosen from the preferred list as aforementioned. Such elements may comprise a recognition site for a CP coding sequence. In other preferred embodiments of the present invention, the producer vector may lack certain coding sequences that facilitate sufficient expression of functional MP (and/or replicase coding sequences). In these embodiments of the present invention, the producer vector may or may not lack coding sequences sufficient for expression of a functional CP.

In another particular embodiment of the present invention, a helper vector is also prepared and is intended to trans-complement the producer vector with its missing components that are needed to enable systemic infection of a plant host. For example, in certain preferred embodiments of the present invention, said helper vectors may include a functional CP coding sequence component. These helper vectors are suitable for trans-complementing a producer vector lacking a functional CP coding sequence component.

The helper vector may lack one or more viral coding component that codes for a replicase or movement protein that is essential for sufficient systemic infection of a plant host when such viral coding component is not also missing in the producer vector. The helper vector may include a GOI which may be identical of different from the GOI in the producer vector. In such cases it may be desirable to use a helper vector that is engineered to be defective for systemic infection because it lacks one or more essential cis-acting coding sequences to minimize spread of the helper vector to non-target plants.

According to the embodiments of the present invention, the helper vector may (but not necessarily) include a cell-to-cell movement coding sequence and/or may lack one or more replicase coding sequences. In these particular embodiments of the present invention, in which the helper vector does not include a cell-to-cell movement coding sequence, such a coding sequence component should be included in the producer vector.

Complete inventive vectors of the present invention, include all essential components necessary for successful systemic infection of a plant host and heterologous expression of a GOI. Different vectors or elements may be derived from one particular plant virus or from multiple plant viruses. In fact, as part of the present invention, it may often be desirable to prepare said inventive vectors from elements of different plant viruses in order to take full advantage of unique viral characteristics of the plant viruses being used (e.g. host range, activity level of promotors, virion dimensions, etc.).

It will be appreciated that this general principle may be applied to any viral vector system comprising trans-complementing vector systems. As will be appreciated by those skilled in the art, that the vector system as presented herein this invention is appropriate for use in accordance with the present invention, so long as the vector set includes a producer vector that is incapable of systemic viral infection (i.e., lacking one or more functional replication protein coding sequence, MP coding sequence, or CP coding sequence components) and a helper vector that facilitates the function or functions lacking in the producer vector. It is to understood that in all embodiments of the present invention, no individual vector, producer or helper, is capable of facilitating systemic viral infection but, as a set, one or both of the vectors is competent for such infection and expression of one or more GOIs.

The inventive vector sets of the present invention, thus diminish the risk of GOI expression in an unintended (non-target) plant host, regardless of the plant species being the same as the target plant. The likelihood that both producer and helper vectors will co-infect an unintended plant host is thus greatly reduced.

The inventive vectors used herein refer to a nucleic acid assembly which is capable of directing the transient expression of an exogenous GOI. The expression vector may include a promoter which is operably linked to the exogenous GOI, restriction endonuclease sites, nucleic acids that encode one or more selection markers, and other nucleic acids useful in the practice of recombinant technologies. Preferably, the expression vector used in step a) comprises: prokaryotic DNA elements encoding an origin of bacterial replication and an antibiotic resistance gene; at least one heterologous nucleotide sequence coding for the aforementioned fusion proteins according to the invention operatively linked to a strong promoter, preferably a 35S promoter; more preferably a double-enhanced 35S promoter, a silencing inhibitor, preferably p19; and elements that control the processing of transcripts, such as termination/polyadenylation sequences, preferably the Tnos sequence.

Numerous plant functional expression promoters and enhancers which can be either tissue specific, developmentally specific, constitutive or inducible can be utilized in conjunction with the constructs of the present invention. As used herein in the specification and in the claims section that follows the phrase “plant promoter” or “promoter” includes a promoter which can direct gene expression in plant cells (including DNA containing organelles, more specifically the protoplast). Such a promoter can be derived from a plant, bacterial, viral, fungal or animal origin.

Such a promoter can be constitutive, i.e., capable of directing high level of gene expression in a plurality of plant tissues, tissue specific, i.e., capable of directing gene expression in a particular plant tissue or tissues, inducible, i.e., capable of directing gene expression under a stimulus, or chimeric, i.e., formed of portions of at least two different promoters. The plant promoter employed can be a constitutive promoter, a tissue specific promoter, an inducible promoter or a chimeric promoter. Examples of constitutive plant promoters include, without being limited to, CaMV35S and CaMV19S promoters, FMV34S promoter, sugarcane bacilliform badnavirus promoter, CsVMV promoter, Arabidopsis ACT2/ACT8 actin promoter, Arabidopsis ubiquitin UBQ1 promoter, barley leaf thionin BTH6 promoter, and rice actin promoter. Examples of tissue specific promoters include, without being limited to, bean phaseolin storage protein promoter, DLEC promoter, PHS promoter, zein storage protein promoter, conglutin gamma promoter from soybean, AT2S1 gene promoter, ACT11 actin promoter from Arabidopsis, napA promoter from Brassica napus and potato patatin gene promoter.

Generally, the inventive vectors described in the present invention are prepared by altering an existing plant virus genome while preserving viral function but simplify the ease of genetic manipulation, for example by removing particular genes and/or by disrupting or substituting particular sequences so as to inactivate or replace them. Analogously, when an inventive vector is found to affirmatively express a particular GOI or activity, it is not necessary that the relevant gene has to be identical to the corresponding gene found in nature. For instance, it is readily apparent that a CP sometimes can tolerate small deletions (Satyanarayana et al., 2014) and still facilitate systemic movement. Thus, so long as any particular protein is functional, it may be used in accordance with the present invention. On the other hand, large deletions are (e.g., more than 20 amino acids) are not favored and should be avoided.

Very high sequence identities with the corresponding native proteins are generally preferred. Typically, the coding sequences expressed in the present invention may show at least 50%, preferably 60%, 70%, 80%, 90%, 91%, 92%, 93%, 94%, 95%, 96%, 97%, 98%, or 99% sequence identity with the corresponding native protein. Consecutively, viral proteins of the present invention should typically show 100% identity with critical functional components of the relevant native viral protein. However, it is duly noted that in case a number of amino acid changes in any particular protein to improve its functionally individually or of other proteins in the assembly complex can be made without significantly affecting the functional activity and/or various other properties of the protein such as stability etc. In particular, many proteins tolerate conservative amino acid changes (i.e., “conservative substitution”) at many different positions without significant reduction in activity or stability. These substitutions are well known in the art and represents one approach to obtaining polypeptide having similar or substantially similar properties to those of a given polypeptide while altering the amino acid (AA) sequence. In amino acid sequences or “variants” thereof as defined herein, amino acids are denoted by single-letter symbols.

These single-letter symbols and three-letter symbols are well known to the person skilled in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (lie) is isoleucine, K (Lys) is lysine, L (Leu) is leucine, M (Met) is methionine, N (Asn) is asparagine, P (Pro) is proline, Q (Gin) is glutamine, R (Arg) is arginine, S (Ser) is serine, T (Thr) is threonine, V (Vai) is valine, W (Trp) is tryptophan, and Y (Tyr) is tyrosine.

In general, amino acids have been classified and divided into groups according to I) charge (positive, negative, or uncharged), II) volume and polarity, and III) Grantham's physico-chemical distance, and combinations of these. For example, amino acids may be divided into 6 categories based on volume and polarity: special (C), neutral and small (A, G, P, S, T), polar and relatively small (N, D, Q, E), polar and relatively large (R, H, K), non-polar and relatively small (I, L, M, V), and non-polar and relatively large (F, W, Y). A conservative AA substitution may be defined as one that replaces one AA with an AA in the same group. Thus, a plethora of functionally equivalent proteins may be derived by introducing on or more conservative AA substitutions in any given viral protein.

Preferably the expression constructs above are present in a vector, and preferably it comprises border sequences which permit the transfer and integration of the expression cassette into the organism genome. Preferably the construct is a plant binary vector. Preferably the binary transformation vector is based on pPZP (Hajdukiewicz, et al. 1994). Other example constructs include pBin19 (see Frisch, D. A., L. W. Harris-Haller, et al. (1995). “Complete Sequence of the binary vector Bin 19.” Plant Molecular Biology 27: 405-409).

As described herein, the invention may be practiced by moving an expression cassette with the requisite components into an existing pBin expression cassette, or in other embodiments a direct-cloning pBin expression vector may be utilised. Preferably the binary vectors include the ColEl origin of replication, although plasmids containing other replication origins that also yield high copy numbers (such as pRi-based plasmids, Lee and Gelvin, 2008) may also be preferred, especially for transient expression systems. If desired, selectable genetic markers may be included in the construct, such as those that confer selectable phenotypes such as resistance to antibiotics or herbicides (e.g. kanamycin, hygromycin, phosphinotricin, chlorsulfuron, methotrexate, gentamycin, spectinomycin, imidazolinones and glyphosate).

### Plant Hosts

It is to be understood that generally any plant that is susceptible to viral infection may be used in accordance with the present invention. In general, it is desirable to utilize plant hosts that are amendable to sufficient biomass growth under defined (and/or confined) conditions, for example in a greenhouse and/or in aqueous systems. It may also be desirable to select plants that are not typically consumed by human beings or domesticated animals and/or animals that are not typically part of the human food chain, so that they may be grown outside without concern that the expressed GOI may be undesirably ingested.

In other situations on the other hand, it may be desirable to employ edible plants. Often, a plant host is chosen according to the GOI to be expressed. When a GOI is desired to be produced in high yields (as will often be the case), it is desirable to select plant hosts with relatively high biomass (e.g., tobacco, which has the additional advantages of I) being highly susceptible to viral infection, II) short growing periods, and III) is not in the human food chain).

It is generally understood that “plant hosts” means any single- or multi-celled organism or a cell, tissue, organ, part or propagation material (such as seeds or fruit) of same which is capable of photosynthesis. Included for the purpose of the invention are all genera and species of higher and lower plants of the Plant Kingdom. Annual, perennial, monocotyledonous and dicotyledonous plants are preferred.

The term includes the mature plants, seed, shoots and seedlings and their derived parts, propagation material (such as seeds or microspores), plant organs, tissue, protoplasts, callus and other cultures, for example cell cultures, and any other type of plant cell grouping to give functional or structural units. Mature plants refer to plants at any desired developmental stage beyond that of the seedling. Seedling refers to a young immature plant at an early developmental stage. Annual, biennial, monocotyledonous and dicotyledonous plants are preferred host organisms for the generation oftransgenic plants.

The expression of genes is furthermore advantageous in all ornamental plants, useful or ornamental trees, flowers, cut flowers, shrubs or lawns. Plants which may be mentioned by way of example but not by limitation are angiosperms, bryophytes such as, for example, Hepaticae (liverworts) and Musci (mosses); Pteridophytes such as ferns, horsetail and club mosses; gymnosperms such as conifers, cycads, ginkgo and Gnetatae; algae such as Chlorophyceae, Phaeophpyceae, Rhodophyceae, Myxophyceae, Xanthophyceae, Bacillariophyceae (diatoms), and Euglenophyceae.

Most preferred are plants which are not used for food or feed purpose such as Arabidopsis thaliana, or preferably Nicotiana tabacum, or most preferably Nicotiana benthamiana. Alternatively, plants which are used for food or feed purposes can be used as well, such as the families of the Leguminosae such as pea, alfalfa and soya; Gramineae such as rice, maize, wheat, barley, sorghum, millet, rye, triticale, or oats; the family of the Umbelliferae, especially the genus Daucus, very especially the species carota (carrot) and Apium, very especially the species Graveolens dulce (celery) and many others; the family of the Solanaceae, especially the genus Lycopersicon, very especially the species esculentum (tomato) and the genus Solanum, very especially the species tuberosum (potato) and melongena (eggplant), and the genus Capsicum, very especially the species annuum (peppers) and many others; the family of the Leguminosae, especially the genus Glycine, very especially the species max (soybean), alfalfa, pea, lucerne, beans or peanut and many others; and the family of the Cruciferae (Brassicacae), especially the genus Brassica, very especially the species napus (oil seed rape), campestris (beet), oleracea cv Tastie (cabbage), oleracea cv Snowball Y (cauliflower) and oleracea cv Emperor (broccoli); and of the genus Arabidopsis, very especially the species thaliana and many others; the family of the Compositae, especially the genus Lactuca, very especially the species sativa (lettuce) and many others; the family of the Asteraceae such as sunflower, Tagetes, lettuce or Calendula and many other; the family of the Cucurbitaceae such as melon, pumpkin/squash or zucchini, and linseed. Further preferred are cotton, sugar cane, hemp, flax, chillies, and the various tree, nut and wine species.

The system of the present invention may be employed to infect, and/or to express a GOI in plant hosts at any stage of development including mature plants, seedlings, sprouts, and seeds. The system may be employed to infect any part of a plant (e.g., roots, leaves, stems, etc.).

### GOI

The embodiments of the present invention may be used to deliver to and/or express in plant hosts any particular GOI such as enzymes, antibodies, hormones, cytokines, regulatory factors, structural proteins, or any other GOI. Encoded proteins may be naturally-occurring proteins, or may be designed or engineered proteins, including for instance fusion proteins (e.g., fusion proteins incorporating part or all of a plant virus protein such as MP or CP).

In certain embodiments of the invention the polynucleotide of interest comprises a portion encoding a tag, e.g., a 6x-His tag, GST, MBP, CBP, Intein-CBD, Streptavidin/Biotin-based tags, HA tag, Myc tag, FLAG tag, and the like. Such tags may conveniently simplify the isolation and/or purification of the protein in question and can be either incorporated into the C- or N-terminal ends. In certain embodiments of the invention the tag is a cleavable tag (e.g., a tag cleavable by a protease such as thrombin) so that the tag can readily be removed after purification, resulting in a protein with native type sequence.

In some instances, it may be desirable to utilize the inventive system to express more than one GOI in the same host plant (e.g., using two different producer vectors, inserting two different GOIs into one producer vector, or inserting one GOI into the producer vector and one GOI into the helper vector), for example in order to produce a multimeric protein or to simultaneously produce two or more different proteins at once).

The system of the present invention can be utilized for the heterologous expression of GOIs, protein-coding or not, of either prokaryotic or eukaryotic origin and generally, encompass the following pharmaceutical proteins of interest, but not limited to, hormones (insulin, thyroid hormone, catecholamines, gonadotropines, trophic hormones, prolactin, oxytocin, dopamine, bovine somatotropin, leptins and the like), growth hormones (e.g., human grown hormone), growth factors (e.g., epidermal growth factor, nerve growth factor, insulin-like growth factor and the like), growth factor receptors, cytokines and immune system proteins (e.g., interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, selectins, homing receptors, T cell receptors, immunoglobulins, soluble major histocompatibility complex antigens, immunologically active antigens such as bacterial, parasitic, or viral antigens or allergens), auto antigens, antibodies), enzymes (tissue plasminogen activator, streptokinase, cholesterol (biosynthetic or degradative), steriodogenic enzymes, kinases, phosphodiesterases, methylases, de-methylases, dehydrogenases, cellulases, proteases, lipases, phospholipases, aromatases, cytochromes, adenylate or guanylate cyclases, neuramidases and the like), receptors (steroid hormone receptors, peptide receptors), binding proteins (steroid binding proteins, growth hormone or growth factor binding proteins and the like), transcription and translation factors, oncoproteins or proto-oncoproteins (e.g., cell cycle proteins), muscle proteins (myosin or tropomyosin and the like), myeloproteins, neuroactive proteins, tumor growth suppressing proteins (angiostatin or endostatin, both of which inhibit angiogenesis), anti-sepsis proteins (bactericidal permeability-increasing protein), structural scleroproteins (such as collagen, fibroin, fibrinogen, elastin, tubulin, actin, and myosin), blood proteins (thrombin, serum albumin, Factor VII, Factor VIII, insulin, Factor IX, Factor X, tissue plasminogen activator, Protein C, von Willebrand factor, antithrombin III, glucocerebrosidase, erythropoietin granulocyte colony stimulating factor (GCSF) or modified Factor VIII, anticoagulants such as Protein S, and huridin) and a variety of vaccine components that may be formulated in a vaccine such as viral coat proteins, viral G proteins, microbial cell wall proteins, microbial toxin proteins, tumor-specific antigens, nucleoproteins, nucleocapsids, and the like.

Also, a large number of enzymes that modify protein or small molecule substrates (e.g., kinases, hydrolases, transferases, etc.), proteins for use in diagnostic or research reagents, nutritionally relevant or total parenteral nutrition (TPN products, are known in the art and can desirably be expressed in plant hosts using the inventive systems described herein.

Introducing Vectors into Plants and Quantification of Transgenes

It is to be understood that the vectors of present invention can be introduced into a plant cell by one or more of hand inoculations where drops of the preparation are put onto the surface of a leaf and gently rubbed; mechanized inoculations of plants where plant bed inoculations are performed by spraying the vector solution onto the plants (which can have cut leaves); high pressure spray of single leaves which provides single plant inoculations by spraying the leaves with a narrow, directed spray, and vacuum infiltration where the inoculations may be accomplished by subjecting a plant host to substantially vacuum pressure environment in order to facilitate infection. Transfer of the vectors of the present invention preferably happens through the use of Agrobacterium which refers to a soil-borne, Gram-negative, rod-shaped phytopathogenic bacterium which causes crown gall. The term “Agrobacterium” includes, but is not limited to, the strains Agrobacterium tumefaciens (which typically causes crown gall in infected plants), and Agrobacterium rhizogenes (which causes hairy root disease in infected host plants). Infection of a plant cell with Agrobacterium generally results in the production of opines (e.g., nopaline, agropine, octopine, etc.) by the infected cell. Thus, Agrobacterium strains which cause production of nopaline (e.g., strain LBA4301, C58, A208) are referred to as “nopaline-type” Agrobacteria; Agrobacterium strains which cause production of octopine (e.g., strain LBA4404, Ach5, B6) are referred to as “octopine-type” Agrobacteria; and Agrobacterium strains which cause production of agropine (e.g., strain EHA105, EHA101, A281) are referred to as “agropine-type” Agrobacteria.

Various suitable assays can be used to determine expression of the transgene. For example, non-limiting examples include detecting and/or quantifying the presence of transcribed sense or anti-sense strands of the transgene by conventional hybridization assays known in the art (e.g., Northern Blot analysis), amplification procedures (e.g., RT-PCR), and array-based technologies. Expression of transgenes can also be determined by examining the protein product. A variety of techniques are available in the art for protein analysis. Non-limiting examples include radio-immunoassays, ELISA (Enzyme Linked Immuno-radiometric assays), “sandwich” immune-assays, immunoradiometric assays, in situ immune-assays (using e.g., colloidal gold, enzyme or radioisotope labels), Western Blot analysis, immunoprecipitation assays, immunofluorescent assays, SDS-PAGE.

It is generally understood by those skilled in the art that determining the protein level involves; I) providing a biological sample containing polypeptides; and II) measuring the amount of any immune-specific binding that occurs between an antibody reactive to the transgene product and a component in the sample, in which the amount of immune-specific binding indicates the level of expressed proteins. Antibodies that specifically recognize and bind to protein products of the transgene are required for immune-assays. These may be purchased from commercial vendors or generated and screened using methods well known in the art.

The sample of test proteins can be prepared by homogenizing the eukaryotic transformant (e.g. cells of a plant host) or their progenies made therefrom (in case of stable transformation), and optionally solubilizing the test protein using any suitable detergent. Results obtained using any such assay on a sample from a transformed host, transiently or stably, is compared with those from a non-transformed source as a control.

Isolation and/or Formulation of Expressed Goi Products

In many embodiments of the present invention, it will be desirable to isolate polynucleotide expression products from the plant tissues that express them. It may also be desirable to formulate such isolated products for their intended use (e.g., as a pharmaceutical or diagnostic agent, or as a reagent, etc.). In other embodiments, it will be desirable to formulate the products together with some or all of the plant tissues that express them.

Where it is desirable to isolate the expression product from some or all of the plant tissue that expresses it, any available purification techniques may be employed. Those of ordinary skill in the art are familiar with a wide range of fractionation and separation procedures. Often, it will be desirable to render the product more than about 50%, preferably more than about 60%, 70%, 80%, 85%, 90%, 91%, 92%, 93%, 94%, 95%, 96%, 97%, 98%, or 99% pure.

Where it is desirable to formulate the product together with the plant material (e.g., for example an oral vaccine formulation when properly formulated), it will often be desirable to have utilized a plant that is not toxic to the relevant recipient (e.g., a human or other animal). Relevant plant tissue (e.g., leaves) may simply be harvested and processed according to techniques known in the art, with due consideration to maintaining activity of the expressed product. In certain embodiments of the invention, it is desirable to have expressed the GOI in an edible plant (and, specifically in edible portions of the plant) so that the material can subsequently be eaten.

Where the polynucleotide encodes or produces a therapeutic agent, it may be formulated according to known techniques. For example, an effective amount of a pharmaceutically active product can be formulated together with one or more organic or inorganic, liquid or solid, pharmaceutically suitable carrier materials. A pharmaceutically active product produced according to the present invention may be employed in dosage forms such as tablets, capsules, troches, dispersions, suspensions, solutions, capsules, creams, ointments, aerosols, powder packets, liquid solutions, solvents, diluents, surface active agents, isotonic agents, thickening or emulsifying agents, preservatives, and solid bindings, as long as the biological activity of the protein is not destroyed by such dosage form.

Materials that can serve as pharmaceutically acceptable carriers include, but are not limited to sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening agents, flavoring agents, and perfuming agents, preservatives, and antioxidants can also be present in the composition, according to the judgment of the formulator and in a composition for the use in accordance with embodiments of the present invention, the carrier and/or excipient may comprise an encapsulating material for at least temporarily encapsulating the compound.

A composition for parenteral injection for the use in accordance with embodiments may be desired in the form of (e.g., may consist of) a sterile aqueous or nonaqueous solution, a dispersion, a suspension and/or an emulsion, and/or a sterile powder for reconstitution into a sterile injectable solution or dispersion prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

A desired composition for the use in accordance with embodiments of the present invention may also contain adjuvants such as, but not limited to, preservatives, wetting agents, emulsifying agents, and dispersing agents. A composition for the use in accordance with embodiments of the present invention may include isotonic agents such as sugars, sodium chloride, and the like.

Where prolonged absorption in an injectable pharmaceutical form is desired, inclusion of agents which delay absorption such as aluminum monostearate and gelatin can be used in accordance with embodiments of the present invention. In cases where a prolonged effect of the composition is desirable by slowing the absorption of the composition when subcutaneously or intramuscularly injected, the composition may comprise a liquid suspension of a crystalline or amorphous material having a poor water solubility. The rate of absorption of the drug may then depend on its rate of dissolution which, in turn, can depend on crystal size and crystalline form. Alternatively, delayed absorption of a parenterally administered drug form may be accomplished by dissolving or suspending the drug in an oil vehicle. Injectable depot forms may be made by forming micro-encapsuled matrices of the drug in biodegradable polymers such as polylactide-polyglycolide. Depending on the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

The composition for a use in accordance with embodiments of the present invention may be provided in a solid dosage form for oral administration such as, but not limited to, capsules, tablets, pills, powders, and granules. In such solid dosage forms, the active compounds may be mixed with at least one excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form can also comprise buffering agents. Solid compositions of a similar type can also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings, such as extended-release, sustained-release, delayed release and immediate-release coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. The active compounds can also be in micro-encapsulated form, if appropriate, with one or more of the above-mentioned excipients.

Liquid dosage forms for oral administration may include, but are not limited to, pharmaceutically acceptable emulsions, solutions, suspensions, syrups and elixirs. In addition to the active compounds, the liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Those skilled in the art will also appreciate that a particularly preferred method of obtaining the desired pharmaceutically active products is by extraction. Infected plants may be extracted to remove the desired products from the residual biomass, thereby increasing the concentration and purity of the product. Plants may also be extracted in a buffered solution. For example, the fresh harvested plants may be transferred into an amount of ice-cold water at a ratio of one to one by weight that has been buffered with, e.g., phosphate buffer. Protease inhibitors can also be added as required. The plants can be disrupted by vigorous blending or grinding while suspended in the buffer solution and the extracted biomass removed by filtration or centrifugation. The transgene product carried in solution can be further purified by additional steps or converted to a dry powder by freeze-drying or precipitation. Extraction can also be carried out by pressing. Live plants can also be extracted by pressing in a press or by being crushed as they are passed through closely spaced rollers. The fluids expressed from the crushed plants are collected and processed according to methods well known in the art. Extraction by pressing allows the release of the products in a more concentrated form. However, the overall yield of the product may be lower than if the product were extracted in solution.

The disclosure of all references cited herein, in as much as it may be used by those skilled in the art to carry out the present invention, is hereby specifically incorporated herein by cross-reference.

### Explementary Experiments and Results of the Inventive Vector System

The inventor has developed a trans-complementary vector system that may readily infect a wide range of plant hosts and yet pose little to no risk of infectious spread. The vector system of the present invention as per exemplification include components of components of a single plant virus or components of different plant viruses.

### Construction of a GFP-Expressing Trans-Complementary TRV-Vector

This trans-complementary system includes components from Tobacco Rattle Virus (TRV). FIG. 2 shows the genome organization of TRV RNA 1 and RNA 2. TRV is a type species of the genus Tobravirus, family Virgaviridae (Carstens, 2010; Robinson, 2005), and is a bipartite, positive-sense single-stranded RNA virus composed of straight tubular particles of two predominant lengths, the longer about 190 nm and the shorter 50 to 115 nm, and a genome consisting of RNA-1 and RNA-2. RNA1 encodes two replicases and one cysteine-enriched protein, which is sufficient for replication and movement within the plant even without RNA2. One coat protein (CP) and two non-structural proteins encoded by RNA2 allow virion formation and nematode-mediated transmission across plants (Ratcliff et al., 1999; Liu et al., 2002).

TRV has a particularly wide host range, infecting more than 100 species in nature and more than 400 species when tested in the glasshouse, although not all of these infections are systemic (S. A. MacFarlane, in Encyclopedia of Virology (Third Edition), 2008).

FIG. 3 Exemplifies a schematic diagram of a replication-competent, GFP-expressing trans-complementary TRV-vector system. Following agroinfiltration of plants, the sequences between the left border (LB) and the right border (RB) of the plasmid vectors mediate gene transferfrom agrobacteria into a chromosome ofa host plant cell. Instead of supplying the full vector as a linear DNA molecule, we use different agrobacteria to deliver various modules of the viral vector and of the gene of interest. Those components are assembled inside a plant cell with the help of a site-specific recombinase (Bxb1 serine integrase). The resulting DNA is transcribed and spliced, thus removing undesired elements such as recombination sites and leading to the creation of a perfect gene/vector sequence within a fully functional infective replicon. Bxb1 Int-mediated recombination uses a minimal attachment site of 48-bp attP (attachment Phage) and a cognate 38-bp site attB (attachment Bacterial) between the donor and recipient DNAs. The attachment sites B and P represent four half-sites. After Bxb1 recombination, a half-site from each remains, yielding two 43-bp sites (attL and attR, Left and Right) flanking the inserted DNA. Bxb1 Int requires no other secondary factors and under these conditions provides irreversible unidirectional integration of donor DNAs.

The 5′ producer vector consists of a modified version of the TRV RNA-1 genome (WD Hamilton, 1987). The resulting producer vector contains, in sequential order, a 754-bp chimeric constitutive promoter with a doubled CaMV 35S enhancer element (Plant Mol Biol. 21:415, 1993; Biotechnol Lett. 29:1793, 2007), a 5121-bp RNA-dependent RNA polymerase (RdRp) which encodes helicase (134 kDa), polymerase (194 kDa) both jointly known as the replicase (Uniprot: P05080, 194K_TRVSY—release number 2014_04), a 756-bp movement protein (MP) (Uniprot: P69473, V29K_TRVSY—release number 2014_04), a 516-bp RNA silencing suppressor (P19) which replaces the native but weaker 16 kDa suppressor of silencing of TRV (Uniprot: P11690, P19_TBSVC—release number 2013_10), a 63-bp apoplast-targeting signal peptide derived from Arabidobsis thaliana 2S seed storage protein 1 (Uniprot: P15457, 2SS1_ARATH—release number 2014_04), a 190-bp intron sequence of castor bean catalase (Uniprot: Q01297 CATA1_RICCO—release number 2014_02), a 48-bp attP(Bxb1) minimal attachment site as one of the two target sites of the Bxb1 serine integrase, a self-cleaving 43-bp hammerhead ribozyme to increase virus infectivity, and finally the 254-bp Nopaline synthase terminator (T-nos) and poly(A) signal from Agrobacterium tumefaciens (A. tumefaciens).

The 3′ helper vector consists of a modified version of TRV RNA-2 genome (WD Hamilton, 1987). The resulting helper vector contains in sequential order, a cognate 38-bp attB(Bxb1) site as one of the two target sites of the Bxb1 serine integrase, a 190-bp intron sequence of castor bean catalase, the 726-bp GOI coding sequence “GFP” (Uniprot: P42212, GFP_AEQVI—release number 2014_04) which is N-terminally fused with endoplasmic reticulum (ER) retention signal (KDEL), the 615-bp coat protein of TRV RNA-2 (Uniprot: P05072, COAT_TRVTC—release number 2013_10), flanked with the 708-bp 3′ untranslated region (3′UTR) of TRV RNA-2 to increase mRNA stability and regulate translational efficiency, and finally the 254-bp T-nos from A. tumefaciens.

The integrase vectors contains in sequential order, a 754-bp chimeric constitutive promoter with a doubled CaMV 35S enhancer element (Plant Mol Biol. 21:415, 1993; Biotechnol Lett. 29:1793, 2007), a 1500-bp Bxb1 serine integrase coding sequence (Uniprot: Q9B086, Q9B086_BPMB1—release number 2014_05), a 45-bp nuclear localization signal derived from Mitogen-activated protein kinase NPK1 (Uniprot: Q40541, NPK1_TOBAC—release number 2014_04), and finally the 254-bp T-nos from A. tumefaciens.

The producer vector lacks long distance mobility and so, if plants are inoculated with this vector alone, its infection is limited to local tissues (i.e., to cells within the initially infected leaf). This replication-competent producer vector is administered together with the separate helper vector bearing a functional CP coding sequence. Preferably, transcripts of these two vectors are mixed with one another and are mechanically applied to plant leaves. Replication of the TRV RNA2 helper vector provides CP, which supports the long distance movement of both vectors into inoculated leaves. Both the producer- and helper vector are mixed with the integrase vector and so, the components of the producer- and helper vector are assembled inside a plant cell in trans with the help of a site-specific recombinase (Bxb1 serine integrase).

The resulting DNA is transcribed and spliced, thus removing undesired elements such as recombination sites and leading to the creation of a perfect gene/vector sequence within a fully functional infective replicon.

The two genes involved in nematode transmission of TRV RNA-2 (2b and 2c) have been removed. Preferably, integration of the vectors into the host genome is avoided, so that transgenic plants are not produced, and the risk that genetic alterations are introduced into the environment is minimized.

The producer-, helper, and integrase fragments were cloned between the T-DNA left and right borders of an improved binary Agrobacterium vector termed “pBINPLUS” which is based on the popular pBIN19 vector. Improvements over pBIN19 include location of the selectable marker gene at the left T-DNA border, a higher copy number in E. coli, and two rare restriction sites around the multiple cloning site for easier cloning and analysis of T-DNA insertions in plant genomes.

SEQ ID No. 1: T-DNA region of the producer vector (FIG. 3),
wherein sequence segments have the following function:

| | | |
|---|---|---|
| Nucleotides | 1 to 25 | T-DNA left border repeat |
| Nucleotides | 26 to 779 | Double CaMV 35S |
| Nucleotides | 780 to 5900 | TRV RNA-1 RdRP 5′end |
| Nucleotides | 5901 to 6659 | TRV RNA-1 movement protein |
| Nucleotides | 6660 to 7176 | P19 silencing surpressor |
| Nucleotides | 7177 to 7240 | Apoplast-targeting signal peptide |
| Nucleotides | 7241 to 7421 | Castor bean catalase intron |
| Nucleotides | 7422 to 7470 | AttP recombination site (Bxb1 |
| | | serine integrase) |
| Nucleotides | 7471 to 7514 | Hammerhead ribozyme |
| Nucleotides | 7515 to 7769 | Nos terminator |
| Nucleotides | 7770 to 7795 | T-DNA right border repeat |

| | |
|---|---|
| 1 | ACCGTCCTAT ATAACACCAC ATTTGGCCAA CATGGTGGAG CACGACACTC TCGTCTACTC |
| 61 | CAAGAATATC AAAGATACAG TCTCAGAAGA CCAAAGGGCT ATTGAGACTT TTCAACAAAG |
| 121 | GGTAATATCG GGAAACCTCC TCGGATTCCA TTGCCCAGCT ATCTGTCACT TCATCAAAAG |
| 181 | GACAGTAGAA AAGGAAGGTG GCACCTACAA ATGCCATCAT TGCGATAAAG GAAAGGCTAT |
| 241 | CGTTCAAGAT GCCTCTGCCG ACAGTGGTCC CAAAGATGGA CCCCCACCCA CGAGGAGCAT |
| 301 | CGTGGAAAAA GAAGACGTTC CAACCACGTC TTCAAAGCAA GTGGATTGAT GTGAACATGG |
| 361 | TGGAGCACGA CACTCTCGTC TACTCCAAGA ATATCAAAGA TACAGTCTCA GAAGACCAAA |
| 421 | GGGCTATTGA GACTTTTCAA CAAAGGGTAA TATCGGGAAA CCTCCTCGGA TTCCATTGCC |
| 481 | CAGCTATCTG TCACTTCATC AAAAGGACAG TAGAAAAGGA AGGTGGCACC TACAAATGCC |
| 541 | ATCATTGCGA TAAAGGAAAG GCTATCGTTC AAGATGCCTC TGCCGACAGT GGTCCCAAAG |
| 601 | ATGGACCCCC ACCCACGAGG AGCATCGTGG AAAAAGAAGA CGTTCCAACC ACGTCTTCAA |
| 661 | AGCAAGTGGA TTGATGTGAT ATCTCCACTG ACGTAAGGGA TGACGCACAA TCCCACTATC |
| 721 | CTTCGCAAGA CCCTTCCTCT ATATAAGGAA GTTCATTTCA TTTGGAGAGG ACACGCTGAA |
| 781 | TGGCTAATGG AAATTTTAAG CTTTCTCAAC TTCTTAATGT TGATGAAATG TCTGCTGAAC |
| 841 | AAAGATCTCA TTTTTTTGAT CTTATGCTTA CTAAGCCAGA TTGTGAAATT GGACAAATGA |
| 901 | TGCAAAGAGT TGTTGTTGAT AAGGTTGATG ATATGATTAG AGAAAGAAAG ACTAAGGATC |
| 961 | CAGTTATTGT TCATGAAGTT CTTTCTCAAA AGGAACAAAA TAAGCTTATG GAAATTTATC |
| 1021 | CAGAATTTAA TATTGTTTTT AAGGATGATA AGAATATGGT TCATGGATTT GCTGCTGCTG |
| 1081 | AAAGAAAGCT TCAAGCTCTT CTTCTTCTTG ATAGAGTTCC AGCTCTTCAA GAAGTTGATG |
| 1141 | ATATTGGAGG ACAATGGTCT TTTTGGGTTA CTAGAGGAGA AAAGAGAATT CATTCTTGTT |
| 1201 | GTCCAAATCT TGATATTAGA GATGATCAAA GAGAAATTTC TAGACAAATT TTTCTTACTG |
| 1261 | CTATTGGAGA TCAAGCTAGA TCTGGAAAGA GACAAATGTC TGAAAATGAA CTTTGGATGT |
| 1321 | ATGATCAATT TAGAAAGAAT ATTGCTGCTC CAAATGCTGT TAGATGTAAT AATACTTATC |
| 1381 | AAGGATGTAC TTGTAGAGGA TTTTCTGATG GAAAGAAGAA GGGAGCTCAA TATGCTATTG |
| 1441 | CTCTTCATTC TCTTTATGAT TTTAAGCTTA AGGATCTTAT GGCTACTATG GTTGAAAAGA |
| 1501 | AGACTAAGGT TGTTCATGCT GCTATGCTTT TTGCTCCAGA ATCTATGCTT GTTGATGAAG |
| 1561 | GACCACTTCC ATCTGTTGAT GGATATTATA TGAAGAAGAA TGGAAAGATT TATTTTGGAT |
| 1621 | TTGAAAAGGA TCCATCTTTT TCTTATATTC ATGATTGGGA AGAATATAAG AAGTATCTTC |
| 1681 | TTGGAAAGCC AGTTTCTTAT CAAGGAAATG TTTTTTATTT TGAACCATGG CAAGTTAGAG |
| 1741 | GAGATACTAT GCTTTTTTCT ATTTATAGAA TTGCTGGAGT TCCAAGAAGA TCTCTTTCTT |
| 1801 | CTCAAGAATA TTATAGAAGA ATTTATATTT CTAGATGGGA AAATATGGTT GTTGTTCCAA |
| 1861 | TTTTTGATCT TGTTGAATCT ACTAGAGAAC TTGTTAAGAA GGATCTTTTT GTTGAAAAGC |
| 1921 | AATTTATGGA TAAGTGTCTT GATTATATTG CTAGACTTTC TGATCAACAA CTTACTATTT |
| 1981 | CTAATGTTAA GTCTTATCTT TCTTCTAATA ATTGGGTTCT TTTTATTAAT GGAGCTGCTG |
| 2041 | TTAAGAATAA GCAATCTGTT GATTCTAGAG ATCTTCAACT TCTTGCTCAA ACTCTTCTTG |
| 2101 | TTAAGGAACA AGTTGCTAGA CCAGTTATGA GAGAACTTAG AGAAGCTATT CTTACTGAAA |
| 2161 | CTAAGCCAAT TACTTCTCTT ACTGATGTTC TTGGACTTAT TTCTAGAAAG CTTTGGAAGC |
| 2221 | AATTTGCTAA TAAGATTGCT GTTGGAGGAT TTGTTGGAAT GGTTGGAACT CTTATTGGAT |
| 2281 | TTTATCCAAA GAAGGTTCTT ACTTGGGCTA AGGATACTCC AAATGGACCA GAACTTTGTT |
| 2341 | ATGAAAATTC TCATAAGACT AAGGTTATTG TTTTTCTTTC TGTTGTTTAT GCTATTGGAG |
| 2401 | GAATTACTCT TATGAGAAGA GATATTAGAG ATGGACTTGT TAAGAAGCTT TGTGATATGT |
| 2461 | TTGATATTAA GAGAGGAGCT CATGTTCTTG ATGTTGAAAA TCCATGTAGA TATTATGAAA |
| 2521 | TTAATGATTT TTTTTCTTCT CTTTATTCTG CTTCTGAATC TGGAGAAACT GTTCTTCCAG |
| 2581 | ATCTTTCTGA AGTTAAGGCT AAGTCTGATA AGCTTCTTCA ACAAAAGAAG GAAATTGCTG |
| 2641 | ATGAATTTCT TTCTGCTAAG TTTTCTAATT ATTCTGGATC TTCTGTTAGA ACTTCTCCAC |
| 2701 | CATCTGTTGT TGGATCTTCT AGATCTGGAC TTGGACTTCT TCTTGAAGAT TCTAATGTTC |
| 2761 | TTACTCAAGC TAGAGTTGGA GTTTCTAGAA AGGTTGATGA TGAAGAAATT ATGGAACAAT |
| 2821 | TTCTTTCTGG ACTTATTGAT ACTGAAGCTG AAATTGATGA AGTTGTTTCT GCTTTTTCTG |
| 2881 | CTGAATGTGA AAGAGGAGAA ACTTCTGGAA CTAAGGTTCT TTGTAAGCCA CTTACTCCAC |
| 2941 | CAGGATTTGA AAATGTTCTT CCAGCTGTTA AGCCACTTGT TTCTAAGGGA AAGACTGTTA |
| 3001 | AGAGAGTTGA TTATTTTCAA GTTATGGGAG GAGAAAGACT TCCAAAGAGA CCAGTTGTTT |
| 3061 | CTGGAGATAA TTCTGTTGAT GCTAGAAGAG AATTTCTTTA TTATCTTGAT GCTGAAAGAG |
| 3121 | TTGCTCAAAA TGATGAAATT ATGTCTCTTT ATAGAGATTA TTCTAGAGGA GTTATTAGAA |
| 3181 | CTGGAGGACA AAATTATCCA CATGGACTTG GAGTTTGGGA TGTTGAAATG AAGAATTGGT |
| 3241 | GTATTAGACC AGTIGTTACT GAACATGCTT ATGTTTTTCA ACCAGATAAG AGAATGGATG |
| 3301 | ATTGGTCTGG ATATCTTGAA GTTGCTGTTT GGGAAAGAGG AATGCTTGTT AATGATTTTG |
| 3361 | CTGTTGAAAG AATGTCTGAT TATGTTATTG TTTGTGATCA AACTTATCTT TGTAATAATA |
| 3421 | GACTTATTCT TGATAATCTT TCTGCTCTTG ATCTTGGACC AGTTAATTGT TCTTTTGAAC |
| 3481 | TTGTTGATGG AGTTCCAGGA TGTGGAAAGT CTACTATGAT TGTTAATTCT GCTAATCCAT |
| 3541 | GTGTTGATGT TGTTCTTTCT ACTGGAAGAG CTGCTACTGA TGATCTTATT GAAAGATTTG |
| 3601 | CTTCTAAGGG ATTTCCATGT AAGCTTAAGA GAAGAGTTAA GACTGTTGAT TCTTTTCTTA |
| 3661 | TGCATTGTGT TGATGGATCT CTTACTGGAG ATGTTCTTCA TTTTGATGAA GCTCTTATGG |
| 3721 | CTCATGCTGG AATGGTTTAT TTTTGTGCTC AAATTGCTGG AGCTAAGAGA TGTATTTGTC |
| 3781 | AAGGAGATCA AAATCAAATT TCTTTTAAGC CAAGAGTTTC TCAAGTTGAT CTTAGATTTT |
| 3841 | CTTCTCTTGT TGGAAAGTTT GATATTGTTA CTGAAAAGAG AGAAACTTAT AGATCTCCAG |
| 3901 | CTGATGTTGC TGCTGTTCTT AATAAGTATT ATACTGGAGA TGTTAGAACT CATAATGCTA |
| 3961 | CTGCTAATTC TATGACTGTT AGAAAGATTG TTTCTAAGGA ACAAGTTTCT CTTAAGCCAG |
| 4021 | GAGCTCAATA TATTACTTTT CTTCAATCTG AAAAGAAGGA ACTTGTTAAT CTTCTTGCTC |
| 4081 | TTAGAAAGGT TGCTGCTAAG GTTTCTACTG TTCATGAATC TCAAGGAGAA ACTTTTAAGG |
| 4141 | ATGTTGTTCT TGTTAGAACT AAGCCAACTG ATGATTCTAT TGCTAGAGGA AGAGAATATC |
| 4201 | TTATTGTTGC TCTTTCTAGA CATACTCAAT CTCTTGTTTA TGAAACTGTT AAGGAAGATG |
| 4261 | ATGTTTCTAA GGAAATTAGA GAATCTGCTG CTCTTACTAA GGCTGCTCTT GCTAGATTTT |
| 4321 | TTGTTACTGA AACTGTTCTT NNNAGATTTA GATCTAGATT TGATGTTTTT AGACATCATG |
| 4381 | AAGGACCATG TGCTGTTCCA GATTCTGGAA CTATTACTGA TCTTGAAATG TGGTATGATG |
| 4441 | CTCTTTTTCC AGGAAATTCT CTTAGAGATT CTTCTCTTGA TGGATATCTT GTTGCTACTA |
| 4501 | CTGATTGTAA TCTTAGACTT GATAATGTTA CTATTAAGTC TGGAAATTGG AAGGATAAGT |
| 4561 | TTGCTGAAAA GGAAACTTTT CTTAAGCCAG TTATTAGAAC TGCTATGCCA GATAAGAGAA |
| 4621 | AGACTACTCA ACTTGAATCT CTTCTTGCTC TTCAAAAGAG AAATCAAGCT GCTCCAGATC |
| 4681 | TTCAAGAAAA TGTTCATGCT ACTGTTCTTA TTGAAGAAAC TATGAAGAAG CTTAAGTCTG |
| 4741 | TTGTTTATGA TGTTGGAAAG ATTAGAGCTG ATCCAATTGT TAATAGAGCT CAAATGGAAA |
| 4801 | GATGGTGGAG AAATCAATCT ACTGCTGTTC AAGCTAAGGT TGTTGCTGAT GTTAGAGAAC |
| 4861 | TTCATGAAAT TGATTATTCT TCTTATATGT ATATGATTAA GTCTGATGTT AAGCCAAAGA |
| 4921 | CTGATCTTAC TCCACAATTT GAATATTCTG CTCTTCAAAC TGTTGTTTAT CATGAAAAGC |
| 4981 | TTATTAATTC TCTTTTTGGA CCAATTTTTA AGGAAATTAA TGAAAGAAAG CTTGATGCTA |
| 5041 | TGCAACCACA TTTTGTTTTT AATACTAGAA TGACTTCTTC TGATCTTAAT GATAGAGTTA |
| 5101 | AGTTTCTTAA TACTGAAGCT GCTTATGATT TTGTTGAAAT TGATATGTCT AAGTTTGATA |
| 5161 | AGTCTGCTAA TAGATTTCAT CTTCAACTTC AACTTGAAAT TTATAGACTT TTTGGACTTG |
| 5221 | ATGAATGGGC TGCTTTTCTT TGGGAAGTTT CTCATACTCA AACTACTGTT AGAGATATTC |
| 5281 | AAAATGGAAT GATGGCTCAT ATTTGGTATC AACAAAAGTC TGGAGATGCT GATACTTATA |
| 5341 | ATGCTAATTC TGATAGAACT CTTTGTGCTC TTCTTTCTGA ACTTCCACTT GAAAAGGCTG |
| 5401 | TTATGGTTAC TTATGGAGGA GATGATTCTC TTATTGCTTT TCCAAGAGGA ACTCAATTTG |
| 5461 | TTGATCCATG TCCAAAGCTT GCTACTAAGT GGAATTTTGA ATGTAAGATT TTTAAGTATG |
| 5521 | ATGTTCCAAT GTTTTGTGGA AAGTTTCTTC TTAAGACTTC TTCTTGTTAT GAATTTGTTC |
| 5581 | CAGATCCAGT TAAGGTTCTT ACTAAGCTTG GAAAGAAGTC TATTAAGGAT GTTCAACATC |
| 5641 | TTGCTGAAAT TTATATTTCT CTTAATGATT CTAATAGAGC TCTTGGAAAT TATATGGTTG |
| 5701 | TTTCTAAGCT TTCTGAATCT GTTTCTGATA GATATCTTTA TAAGGGAGAT TCTGTTCATG |
| 5761 | CTCTTTGTGC TCTTTGGAAG CATATTAAGT CTTTTACTGC TCTTTGTACT CTTTTTAGAG |
| 5821 | ATGAAAATGA TAAGGAACTT AATCCAGCTA AGGTTGATTG GAAGAAGGCT CAAAGAGCTG |
| 5881 | TTTCTAATTT TTATGATTGG ATGGAAGATA AGTCTCTTGT TACTCTTAAG AAGAAGACTT |
| 5941 | TTGAAGTTTC TAAGTTTTCT AATCTTGGAG CTATTGAACT TTTTGTTGAT GGAAGAAGAA |
| 6001 | AGAGACCAAA GTATTTTCAT AGAAGAAGAG AAACTGTTCT TAATCATGTT GGAGGAAAGA |
| 6061 | AGTCTGAACA TAAGCTTGAT GTTTTTGATC AAAGAGATTA TAAGATGATT AAGTCTTATG |
| 6121 | CTTTTCTTAA GATTGTTGGA GTTCAACTTG TTGTTACTTC TCATCTTCCA GCTGATACTC |
| 6181 | CAGGATTTAT TCAAATTGAT CTTCTTGATT CTAGACTTAC TGAAAAGAGA AAGAAGGGAA |
| 6241 | AGACTATTCA AAGATTTAAG GCTAGAGCTT GTGATAATTG TTCTGTTGCT CAATATAAGG |
| 6301 | TTGAATATTC TATTTCTACT CAAGAAAATG TTCTTGATGT TTGGAAGGTT GGATGTATTT |
| 6361 | CTGAAGGAGT TCCAGTTTGT GATGGAACTT ATCCATTTTC TATTGAAGTT TCTCTTATTT |
| 6421 | GGGTTGCTAC TGATTCTACT AGAAGACTTA ATGTTGAAGA ACTTAATTCT TCTGATTATA |
| 6481 | TTGAAGGAGA TTTTACTGAT CAAGAAGTTT TTGGAGAATT TATGTCTCTT AAGCAAGTTG |
| 6541 | AAATGAAGAC TATTGAAGCT AAGTATGATG GACCATATAG ACCAGCTACT ACTAGACCAA |
| 6601 | AGTCTCTTCT TTCTTCTGAA GATGTTAAGA GAGCTTCTAA TAAGAAGAAT TCTTCTATGG |
| 6661 | AAAGAGCTAT TCAAGGAAAT GATGCTAGAG AACAAGCTAA TTCTGAAAGA TGGGATGGAG |
| 6721 | GATCTGGAGG AACTACTTCT CCATTTAAGC TTCCAGATGA ATCTCCATCT TGGACTGAAT |
| 6781 | GGAGACTTCA TAATGATGAA ACTAATTCTA ATCAAGATAA TCCACTTGGA TTTAAGGAAT |
| 6841 | CTTGGGGATT TGGAAAGGTT GTTTTTAAGA GATATCTTAG ATATGATAGA ACTGAAGCTT |
| 6901 | CTCTTCATAG AGTTCTTGGA TCTTGGACTG GAGATTCTGT TAATTATGCT GCTTCTAGAT |
| 6961 | TTTTTGGATT TGATCAAATT GGATGTACTT ATTCTATTAG ATTTAGAGGA GTTTCTATTA |
| 7021 | CTGTTTCTGG AGGATCTAGA ACTCTTCAAC ATCTTTGTGA AATGGCTATT AGATCTAAGC |
| 7081 | AAGAACTTCT TCAACTTGCT CCAATTGAAG TTGAATCTAA TGTTTCTAGA GGATGTCCAG |
| 7141 | AAGGAACTGA AACTTTTGAA AAGGAATCTG AAATGGCTAA TAAGCTTTTT CTTGTTTGTG |
| 7201 | CTGCTCTTGC TCTTTGTTTT CTTCTTACTA ATGCTCATTT AAAGATCAAA AAGAGGAAGT |
| 7261 | AAAAGAACCA ATCCTGGGAA AAGAGAAAAA TAAAAAAACT CGAAACTAGA AAGAAATTTG |
| 7321 | ACTAGATAAA AAATTAACTA ACCAATACCA CATTTATAAT GTATCGAAAT TGACTATTAG |
| 7381 | ACTAATGAAA TAAAGCACAC AGATACTACT ACTACTATCA ATGTCGGTTT GTCTGGTCAA |
| 7441 | CCACCGCGGT CTCAGTGGTG TACGGTACAA ACCGGCATGC TGATGAGTCC GTGAGGACGA |
| 7501 | AACGAGTAAG CTCGTCGATC GTTCAAACAT TTGGCAATAA AGTTTCTTAA GATTGAATCC |
| 7561 | TGTTGCCGGT CTTGCGATGA TTATCATATA ATTTCTGTTG AATTACGTTA AGCATGTAAT |
| 7621 | AATTAACATG TAATGCATGA CGTTATTTAT GAGATGGGTT TTTATGATTA GAGTCCCGCA |
| 7681 | ATTATACATT TAATACGCGA TAGAAAACAA AATATAGCGC GCAAACTAGG ATAAATTATC |
| 7741 | GCGCGCGGTG TCATCTATGT TACTAGATCG ACTGTCCTAT ATAACCGCCC ATTTG |

SEQ ID NO. 2: T-DNA region of the helper vector (FIG. 3),
wherein sequence segments have the following function:

| | | |
|---|---|---|
| Nucleotides | 1 to 25 | T-DNA left border repeat |
| Nucleotides | 26 to 63 | Attp recombination site |
| | | (Bxb1 serine integrase) |
| Nucleotides | 64 to 253 | Castor bean catalase intron |
| Nucleotides | 254 to 979 | KDEL N-terminally fused GFP |
| Nucleotides | 980 to 1594 | TRV RNA-2 coat protein |
| Nucleotides | 1595 to 2302 | TRV RNA-2 3'UTR |
| Nucleotides | 2303 to 2556 | Nos terminator |
| Nucleotides | 2557 to 2581 | T-DNA right border repeat |

| | |
|---|---|
| 1 | ACCGTCCTAT ATAACACCAC ATTTGGGCTT GTCGACGACG GCGGTCTCCG TCGTCAGGAT |
| 61 | CATCATTTAA AGATCAAAAA GAGGAAGTAA AAGAACCAAT CCTGGGAAAA GAGAAAAATA |
| 121 | AAAAAACTCG AAACTAGAAA GAAATTTGAC TAGATAAAAA ATTAACTAAC CAATACCACA |
| 181 | TTTATAATGT ATCGAAATTG ACTATTAGAC TAATGAAATA AAGCACACAG ATACTACTAC |
| 241 | TACTATCAAT GTCAAGGATG AACTTATGTC TAAGGGAGAA GAACTTTTTA CTGGAGTTGT |
| 301 | TCCAATTCTT GTTGAACTTG ATGGAGATGT TAATGGACAT AAGTTTTCTG TTTCTGGAGA |
| 361 | AGGAGAAGGA GATGCTACTT ATGGAAAGCT TACTCTTAAG TTTATTTGTA CTACTGGAAA |
| 421 | GCTTCCAGTT CCATGGCCAA CTCTTGTTAC TACTTTTTCT TATGGAGTTC AATGTTTTTC |
| 481 | TAGATATCCA GATCATATGA AGCAACATGA TTTTTTTAAG TCTGCTATGC CAGAAGGATA |
| 541 | TGTTCAAGAA AGAACTATTT TTTTTAAGGA TGATGGAAAT TATAAGACTA GAGCTGAAGT |
| 601 | TAAGTTTGAA GGAGATACTC TTGTTAATAG AATTGAACTT AAGGGAATTG ATTTTAAGGA |
| 661 | AGATGGAAAT ATTCTTGGAC ATAAGCTTGA ATATAATTAT AATTCTCATA ATGTTTATAT |
| 721 | TATGGCTGAT AAGCAAAAGA ATGGAATTAA GGTTAATTTT AAGATTAGAC ATAATATTGA |
| 781 | AGATGGATCT GTTCAACTTG CTGATCATTA TCAACAAAAT ACTCCAATTG GAGATGGACC |
| 841 | AGTTCTTCTT CCAGATAATC ATTATCTTTC TACTCAATCT GCTCTTTCTA AGGATCCAAA |
| 901 | TGAAAAGAGA GATCATATGG TTCTTCTTGA ATTTGTTACT GCTGCTGGAA TTACTCATGG |
| 961 | AATGGATGAA CTTTATAAGA TGGCTGGATC TTATGGAGAA ACTTTTGATG GAAAGATTCT |
| 1021 | TGATGATCTT TCTGGAGCTT GGGTTGAAAA GCATAATTGG TCTGATATTC TTAGAAGACT |
| 1081 | TACTAAGATT AAGTTTGCTC TTCAAGCTGA TAGAGATATG ATTCCAGGAA TTGTTGAAGA |
| 1141 | TCTTTCTACT GAAATTCCAG TTGATGAAAA TACTAGATTT CCATCTGGAA AGGTTTATCA |
| 1201 | TCTTCTTACT AAGGAAATGC TTATGGCTAT TGAAGCTATT CATGCTGCTT CTTCTTTTAA |
| 1261 | GAGAAGAGCT GAAGAAAAGA ATAATGTTAA TCCAAGACAA AGATTTGAAG CTGAATCTTC |
| 1321 | TTCTTCTCAA CTTCCATCTG GAGGACTTGT TGTTAGACCA GCTGCTGGAT CTGGAGATTC |
| 1381 | TTCTCTTGGA GAAGATCTTT TTTCTAATTC TAAGCTTGAT GATGCTTCTA CTGCTTTTCA |
| 1441 | TAAGTCTCTT GCTACTCTTA AGGGATCTAG ACCAAAGGCT GTTGTTCAAA GAACTTTTGA |
| 1501 | AAAGGAATAT TCTCTTAGAT GGACTGCTGC TGCTCCAGTT GCTGCTGCTG GAGGAACTCC |
| 1561 | ACCAGGAGGA AGATCTTGGA CTTGGAATCT TGTTTTCCGG GACCATCTTG ATATTTACAA |
| 1621 | CTTAACTTGT TCTCCCCCTT TCCTGGTGAG CAGTGCGGTT GTAGATGGAC ACTACGCACG |
| 1681 | GGATAAGTTC GTTAGTTTTC AAGGTGTATG TGGGTTCAAT CCGATGTTTC CAGACGTGAA |
| 1741 | TGGTTTGAAG AGTAGTTGGT CTTTAGGTCG TCAACTGGAT GATATACGGT CTCAAAAGAA |
| 1801 | AGAGGTTTCT GGAACAAACC AGGAGCCAAA CTATTACTAC GATGGCGACA CACTAAAACC |
| 1861 | GATCGGTTCA GGTGCGAGCG TTGTAGGAGA GAGAAGGCCG GGATGGAGGT GAATGTCCCG |
| 1921 | AAGACATTAA ACTACGTTCT TTAAGTAGAT CCGTGCCTGA AGTTTTAGGT TCAATTTAAA |
| 1981 | CCTACGAGAT TGACATTCTC GACTGATCTT GATTGATCGG TAAGTCTTTT GTAATTTAAT |
| 2041 | TTTCTTTTTG ATTTTATTTT AAATTGTTAT CTGTTTCTGT GTATAGACTG TTTGAGATCG |
| 2101 | GCGTTTAGGC TCATTGTCTT ACCATAGGGG AACGGACTTT GTTTGTGTTG TTATTTTATT |
| 2161 | TGTATTTTAT TAAAATTCTC AACGATCTGA AAAAGCCTCG CGCTAAGAGA TTGTTGGGGG |
| 2221 | GTGAGTAAGT ACTTTTAAAG TGATGATGGT TACAAAGGCA AAAGGGGTAA AACCCCTCGC |
| 2281 | CTACGTAAGC GTTATTACGC CCGATCGTTC AAACATTTGG CAATAAAGTT TCTTAAGATT |
| 2341 | GAATCCTGTT GCCGGTCTTG CGATGATTAT CATATAATTT CTGTTGAATT ACGTTAAGCA |
| 2401 | TGTAATAATT AACATGTAAT GCATGACGTT ATTTATGAGA TGGGTTTTTA TGATTAGAGT |
| 2461 | CCCGCAATTA TACATTTAAT ACGCGATAGA AAACAAAATA TAGCGCGCAA ACTAGGATAA |
| 2521 | ATTATCGCGC GCGGTGTCAT CTATGTTACT AGATCGACTG TCCTATATAA CCGCCCATTT |
| 2581 | G |

SEQ ID NO. 3: T-DNA region of the integrase vector (FIG. 3),
wherein sequence segments have the following function:

| | | |
|---|---|---|
| Nucleotides | 1 to 25 | T-DNA left border repeat |
| Nucleotides | 26 to 779 | Double CaMV 35S |
| Nucleotides | 779 to 2278 | Bxb1 serine integrase |
| Nucleotides | 2279 to 2323 | Nuclear localization signal |
| Nucleotides | 2324 to 2578 | Nos terminator |
| Nucleotides | 2579 to 2603 | T-DNA right border repeat |

| | |
|---|---|
| 1 | ACCGTCCTAT ATAACACCAC ATTTGGCCAA CATGGTGGAG CACGACACTC TCGTCTACTC |
| 61 | CAAGAATATC AAAGATACAG TCTCAGAAGA CCAAAGGGCT ATTGAGACTT TTCAACAAAG |
| 121 | GGTAATATCG GGAAACCTCC TCGGATTCCA TTGCCCAGCT ATCTGTCACT TCATCAAAAG |
| 181 | GACAGTAGAA AAGGAAGGTG GCACCTACAA ATGCCATCAT TGCGATAAAG GAAAGGCTAT |
| 241 | CGTTCAAGAT GCCTCTGCCG ACAGTGGTCC CAAAGATGGA CCCCCACCCA CGAGGAGCAT |
| 301 | CGTGGAAAAA GAAGACGTTC CAACCACGTC TTCAAAGCAA GTGGATTGAT GTGAACATGG |
| 361 | TGGAGCACGA CACTCTCGTC TACTCCAAGA ATATCAAAGA TACAGTCTCA GAAGACCAAA |
| 421 | GGGCTATTGA GACTTTTCAA CAAAGGGTAA TATCGGGAAA CCTCCTCGGA TTCCATTGCC |
| 481 | CAGCTATCTG TCACTTCATC AAAAGGACAG TAGAAAAGGA AGGTGGCACC TACAAATGCC |
| 541 | ATCATTGCGA TAAAGGAAAG GCTATCGTTC AAGATGCCTC TGCCGACAGT GGTCCCAAAG |
| 601 | ATGGACCCCC ACCCACGAGG AGCATCGTGG AAAAAGAAGA CGTTCCAACC ACGTCTTCAA |
| 661 | AGCAAGTGGA TTGATGTGAT ATCTCCACTG ACGTAAGGGA TGACGCACAA TCCCACTATC |
| 721 | CTTCGCAAGA CCCTTCCTCT ATATAAGGAA GTTCATTTCA TTTGGAGAGG ACACGCTGAA |
| 781 | TGAGAGCTCT TGTTGTTATT AGACTTTCTA GAGTTACTGA TGCTACTACT TCTCCAGAAA |
| 841 | GACAACTTGA ATCTTGTCAA CAACTTTGTG CTCAAAGAGG ATGGGATGTT GTTGGAGTTG |
| 901 | CTGAAGATCT TGATGTTTCT GGAGCTGTTG ATCCATTTGA TAGAAAGAGA AGACCAAATC |
| 961 | TTGCTAGATG GCTTGCTTTT GAAGAACAAC CATTTGATGT TATTGTTGCT TATAGAGTTG |
| 1021 | ATAGACTTAC TAGATCTATT AGACATCTTC AACAACTTGT TCATTGGGCT GAAGATCATA |
| 1081 | AGAAGCTTGT TGTTTCTGCT ACTGAAGCTC ATTTTGATAC TACTACTCCA TTTGCTGCTG |
| 1141 | TTGTTATTGC TCTTATGGGA ACTGTTGCTC AAATGGAACT TGAAGCTATT AAGGAAAGAA |
| 1201 | ATAGATCTGC TGCTCATTTT AATATTAGAG CTGGAAAGTA TAGAGGATCT CTTCCACCAT |
| 1261 | GGGGATATCT TCCAACTAGA GTTGATGGAG AATGGAGACT TGTTCCAGAT CCAGTTCAAA |
| 1321 | GAGAAAGAAT TCTTGAAGTT TATCATAGAG TTGTTGATAA TCATGAACCA CTTCATCTTG |
| 1381 | TTGCTCATGA TCTTAATAGA AGAGGAGTTC TTTCTCCAAA GGATTATTTT GCTCAACTTC |
| 1441 | AAGGAAGAGA ACCACAAGGA AGAGAATGGT CTGCTACTGC TCTTAAGAGA TCTATGATTT |
| 1501 | CTGAAGCTAT GCTTGGATAT GCTACTCTTA ATGGAAAGAC TGTTAGAGAT GATGATGGAG |
| 1561 | CTCCACTTGT TAGAGCTGAA CCAATTCTTA CTAGAGAACA ACTTGAAGCT CTTAGAGCTG |
| 1621 | AACTTGTTAA GACTTCTAGA GCTAAGCCAG CTGTTTCTAC TCCATCTCTT CTTCTTAGAG |
| 1681 | TTCTTTTTTG TGCTGTTTGT GGAGAACCAG CTTATAAGTT TGCTGGAGGA GGAAGAAAGC |
| 1741 | ATCCAAGATA TAGATGTAGA TCTATGGGAT TTCCAAAGCA TTGTGGAAAT GGAACTGTTG |
| 1801 | CTATGGCTGA ATGGGATGCT TTTTGTGAAG AACAAGTTCT TGATCTTCTT GGAGATGCTG |
| 1861 | AAAGACTTGA AAAGGTTTGG GTTGCTGGAT CTGATTCTGC TGTTGAACTT GCTGAAGTTA |
| 1921 | ATGCTGAACT TGTTGATCTT ACTTCTCTTA TTGGATCTCC AGCTTATAGA GCTGGATCTC |
| 1981 | CACAAAGAGA AGCTCTTGAT GCTAGAATTG CTGCTCTTGC TGCTAGACAA GAAGAACTTG |
| 2041 | AAGGACTTGA AGCTAGACCA TCTGGATGGG AATGGAGAGA AACTGGACAA AGATTTGGAG |
| 2101 | ATTGGTGGAG AGAACAAGAT ACTGCTGCTA AGAATACTTG GCTTAGATCT ATGAATGTTA |
| 2161 | GACTTACTTT TGATGTTAGA GGAGGACTTA CTAGAACTAT TGATTTTGGA GATCTTCAAG |
| 2221 | AATATGAACA ACATCTTAGA CTTGGATCTG TTGTTGAAAG ACTTCATACT GGAATGTCTA |
| 2281 | GAAGATGGAA GGAAGAACTT GATGAAGAAC TTCAAAGAAA GAGAGATCGT TCAAACATTT |
| 2341 | GGCAATAAAG TTTCTTAAGA TTGAATCCTG TTGCCGGTCT TGCGATGATT ATCATATAAT |
| 2401 | TTCTGTTGAA TTACGTTAAG CATGTAATAA TTAACATGTA ATGCATGACG TTATTTATGA |
| 2461 | GATGGGTTTT TATGATTAGA GTCCCGCAAT TATACATTTA ATACGCGATA GAAAACAAAA |
| 2521 | TATAGCGCGC AAACTAGGAT AAATTATCGC GCGCGGTGTC ATCTATGTTA CTAGATCGAC |
| 2581 | TGTCCTATAT AACCGCCCAT TTG |

Agroinfiltration Procedure in N. benthamiana

The 3 resulting binary vectors were separately introduced into Agrobacterium tumefaciens strains GV3101, or LBA4404, or LBA4301 through electroporation. The resulting agrobacteria were verified by restriction digestion or PRC of plasmid DNA, grown overnight at 30° C., and used to infiltrate leaves of 5-week old N. benthamiana maintained at 23-25° C. Briefly, the bacteria were pelleted by centrifugation for 5 minutes at 5,000 g and then resuspended in infiltration buffer [10 mM 2-(N-morpholino)ethanesulfonic acid (MES), pH 5.5 and 10 mM MgSO4] to OD600=0.2. The resulting bacterial suspensions were injected by using a syringe without needle into leaves through a small puncture. Plant tissue was harvested at 4 DPI unless otherwise noted.

### RT-PCR Procedure

First strand cDNAs were synthesized from 5 μg total RNA treated with DNasel by using the SuperScript First-Strand Synthesis System for RT-PCR (Invitrogen, Thermo Fisher Scientific). PCR amplification was performed by using the following protocol: initial denaturation at 94° C. for 3 min followed by 35 cycles of denaturation at 94° C. for 30 sec, annealing at 65° C. for 30 sec, elongation at 72° C. for 1 min, and finally a 3-min extension at 72° C.

Isolation of N. benthamiana Mesophyll Protoplasts

Protoplasts were released from leaf tissue by incubation for 12-14 h at 25° C. in the dark in a solution containing 0.5% Cellulase Onozuka R-10 (Duchefa Biochemie), 0.3% Macerozyme R-10 (Duchefa Biochemie), 0.2% Pectolyase Y-23 (Duchefa Biochemie), 0.5 M sucrose, 7 mM CaCl₂), 90 mM glycine, and protease inhibitor cocktail (Roche Life Science). Released protoplasts were filtrated through a tri-layer consisting of cheese-cloth (upper layer), miracloth (middle layer) and a nylon mesh (lower layer) with a 50-μm pore diameter and resuspended in modified W5m solution and centrifuged at 179 xg for purification. For the modified W5m solution, Ca(No₃)₂ was used instead of CaCl₂) to prevent further vacuolar acidification and cell expansion. The total yield of protoplast reached as high as 6.04×10⁷ protoplasts g⁻¹ fresh weight (FW) and the viability of the protoplasts was up to 95%.

Visualization of GFP Leaves expressing GFP were viewed under UV illumination generated by a B-100AP/R lamp (labortechnik). Protoplasts were viewed with a ZEISS Primovert inverted microscope with fluorescence filter sets for GFP (Techspec, excitation filter, 470/40 nm; barrier filter, 525/50 nm). Protoplasts were also viewed with an Axioplan 2 microscope (Zeiss) using GFP filter block.

### Results

Producer-, Helper, and Integrase T-DNAs can be Delivered to the Same Cell from Different Agrobacteria Efficiently

N. benthamiana, infiltration of leaf tissue with an Agrobacterium suspension resulted in transient expression in nearly 100% of the mesophyll cells. To quantify the efficiency of co-delivery of the producer-, helper-, and integrase vectors into the same cell, N. benthamiana leaves were co-infiltrated with a mixture of three different Agrobacterium cultures (producer vector with GV3101, helper vector with LBA4404, and integrase vector with LBA4301) and found that >95% of protoplasts isolated from infected areas expressed GFP (FIG. 4A). Subsequent studies showed that leaving out the integrase construct, no GFP expression was detected (FIG. 4B) and thus integration of the integrase vector is mandatory for functionality of the trans-complementary vector system.

### Transiently Expressed T-DNAs Recombine Efficiently in Plant Cells and can Assemble in Planta

It is known that T-DNAs can recombine after delivery to a plant cell (Vergunst, A. C., Jansen, L. E. & Hooykaas, P. J., 1998). However, to be useful for gene assembly in planta, site-specific recombination of T-DNAs needs to be very efficient. To measure the ability of T-DNAs to recombine under the control of Bxb1 serine integrase, we tested its ability by either co-infiltration of the integrase vector (FIG. 3) with the producer- and helper vector, or without. Co-infiltration with the integrase vector resulted in clearly visible GFP staining of the entire infiltrated area (FIG. 5A), whereas no GFP activity could be detected in the absence of the integrase vector in the control infiltration, thereby showing that site-specific recombination of T-DNAs, in planta assembly, and viral amplification takes place efficiently in the majority of infiltrated cells only by co-infiltration of the integrase vector (FIG. 5B).

Replacement of the TRV RNA-1 K16 Protein with P19 Improved Suppressor of PTGS Functionality

RNA silencing is a host defense mechanism that limits the accumulation and spread of viruses in infected plants. Correspondingly, plant viruses encode suppressors of silencing. In the positive-strand RNA virus Tobacco rattle virus (TRV), the suppressor of silencing is a 16-kDa (16K) protein encoded by RNA1. The suppressor action of the 16K protein is transient and weaker than that of the P19 suppressor, encoded by tomato bushy stunt virus (TBSV). Two trans-complementary vector systems were tested against each other for their ability to suppress PTGS and improve viral amplification and spread. By replacing the original 16K protein of TRV with the P19 suppressor gene from TBSV, PTGS was significantly more suppressed as compared with the original K16 bearing vector system, thereby improving GFP expression significantly (FIG. 6).

Replacement of the TRV RNA-1 K16 Protein with P19 Improved Suppressor of PTGS Functionality

RNA silencing is a host defense mechanism that limits the accumulation and spread of viruses in infected plants. Correspondingly, plant viruses encode suppressors of silencing. In the positive-strand RNA virus Tobacco rattle virus (TRV), the suppressor of silencing is a 16-kDa (16K) protein encoded by RNA1. The suppressor action of the 16K protein is transient and weaker than that of the P19 suppressor, encoded by tomato bushy stunt virus (TBSV). Two trans-complementary vector systems were tested against each other for their ability to suppress PTGS and improve viral amplification and spread. By replacing the original 16K protein of TRV in the producer vector with the P19 suppressor gene from TBSV, PTGS was significantly more suppressed as compared with the original K16 bearing vector system, thereby improving GFP expression significantly (FIG. 6).

### Intron Flanking of Attp/Attb Recombination Sites Improve GFP Expression Rates

Splicing is the process by which introns, the noncoding regions of genes, are excised out of the primary messenger RNA transcript, and the exons (i.e., coding regions) are joined together to generate mature messenger RNA. The latter serves as the template for synthesis of a specific protein. Introns can increase transcript levels by affecting the rate of transcription, nuclear export, and transcript stability. Moreover, introns can also increase the efficiency of mRNA translation. One of the goals of the inventor for designing this system was to improve expression rates and viral amplification and raising the opportunity to remove unwanted sequences by splicing. Therefore, recombination site sequences Attp in the 5′ producer vector, and Attb in the 3′ helper vector were flanked by intron sequences. Three criteria were used for choosing an optimal intron: (i) a size close to the average size of plant introns (100-250 bp), (ii) a low GC content, and (iii) sequences of the splice sites close to the consensus. The first criterion is easy to fulfill, because numerous introns of all sizes are present in genome databases. Therefore, we chose a 190-nt intron from castor bean catalase, which has an average GC distribution content of 13% (FIG. 7A). To respect the second criterion, we chose very T-rich sequences to flank the recombination sequences, and we made sure that no cryptic splice site was present in the intron near the GC-rich sequences of the recombination site. Finally, sequences of the splice sites were modified to fit exactly the consensus for dicot plants.

A set of producer- and helper vectors bearing the intron sequences from castor bean catalase flanked at the Attp/Attb recombination sites, and a set of producer- and helper vectors without the intro sequences were designed for studying the ability of intron inclusion to increase expression rates. The inclusion of sequences from castor bean catalase flanking the recombination sites, clearly improved expression rates significantly (FIGS. 7B, and 7C).

### TRV RNA-2 Helper Vector Complements TRV RNA-1 Producer Vector by Providing GFP Expression Under the Control of CP in Trans to Enable Long Distance Movement

There are two types of virus movement: i) slow, local movement, in which the virus moves from one cell into neighboring cells, and ii) fast, systemic movement, in which the virus moves from an infection site to distant parts of the plant by hitching a ride on the plant's own supply lines (the veins). TRV does not require CP and viral particles for viral long-distance movement in plants and, hence, systemic infection with RNA1 alone can occur (Swanson et al. 2002). Therefore, the vector system was designed in a way that the producer vector is only functionalized by i) providing the GOI and CP for improved systemic infection in trans by the helper vector, and ii) providing the integrase vector to enable in planta assembly of the producer- and helper vector.

An expression experiment was conducted where the inventor clearly shows that providing the CP in trans via the helper vector, enables long distance thus systemic infection of plant material. A helper vector where the CP coding sequence was deleted, clearly showed that only local infection was obtained. Experiment was carried out by one-point local syringe agroinfiltration on both plant groups. Quantitation of transient expression of GFP in plants from both vector systems were analyzed. Top. Individual N. benthamiana leaves infiltrated with (A) A. tumefaciens suspensions bearing the vector system without the CP coding sequence in the helper vector, or (B) A. tumefaciens suspensions bearing the vector system with the CP coding sequence in the helper vector. (C) Non-infiltrated control leaf. Leaves photographed (4 days post infiltration, DPI) under UV light to visualize GFP. Bottom. Micrograms of GFP produced per gram of infiltrated tissue as estimated by GFP fluorescence assay. Labelling same as in top of figure. Plant extracts prepared 4-5 DPI were analyzed with a plate-based GFP fluorescence assay. Three plants of each treatment were analyzed. Samples were analyzed in triplicate, and values averaged. Purified His-6 tagged GFP was used to generate a standard curve (FIG. 8A, and 8B).

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

### BRIEF DESCRIPTIONS OF THE FIGURES

FIG. 1 depicts families and genera of DNA and RNA viruses infecting plants

FIG. 2 depicts the genome organization of TRV

FIG. 3 depicts the construction of a GFP-expressing trans-complementary TRV vector

FIG. 4 depicts a TRV trans-complementary system showing gfp expression in N. benthamia protoplasts

FIG. 5 depicts the successful transient expression of GFP using a TRV trans-complementary system that recombines efficiently in plant cells with the integrase vector

FIG. 6 depicts the improved PTGS suppression by replacement of the original k16 protein of TRV, with the p19 protein from TBSV

FIG. 7 depicts the improved viral amplification and expression of GFP by flanking the recombination sites with castor bean catalase intron sequences

FIG. 8 Depicts the TRV RNA-2 helper vector complementing TRV RNA-1 producer vector by providing GFP expression under the control of CP in-trans to enable long distance movement

FIG. 9A depicts a comparison of healthy and infected N. benthamiana plants grown at 21° C. Not infected (left panel), infected with the producer vector bearing the P19 silencing inhibitor from TBSV (middle), and infected with the producer vector bearing the native K19 silencing inhibitor from TRV (right panel).

FIG. 9B depicts plant health after 10 days inoculation either with the inventive vector versus the well-known py1192 TRV vector. Here control indicates buffer control (0.01 M phosphate buffer).

FIG. 10 shows GFP analysis in N. benthamiana based on GFP and western blot assays. (A) GFP imaging at 3 dpi in N. benthamiana infiltrated with either the trans-complementary vector (TC-TRV) with the native K16 without intron flanking, the TC-TRV vector with the P19 silencing suppressor without intron flanking, or the TC-TRV vector with the P19 silencing suppressor and with intron flanking. (B) GFP protein analysis in N. benthamiana infiltrated as for (A). GFP expression for the TC-TRV vectors was monitored each day from 3 to 7 days post-infiltration (dpi) with Mock (M) and 00 (control Agrobacterium expressing an empty TRV vector) collected at 7 dpi. In each case the upper panels are western blots for GFP detection, while the lower panels show the corresponding Coomassie Brilliant Blue (CB) staining of the gels for loading comparison. The red colored size marker (S) on the left of western blots is 25 kDa, while the size of the intense CB band of a host protein is about 55 kDa as inferred from the size marker on the left.

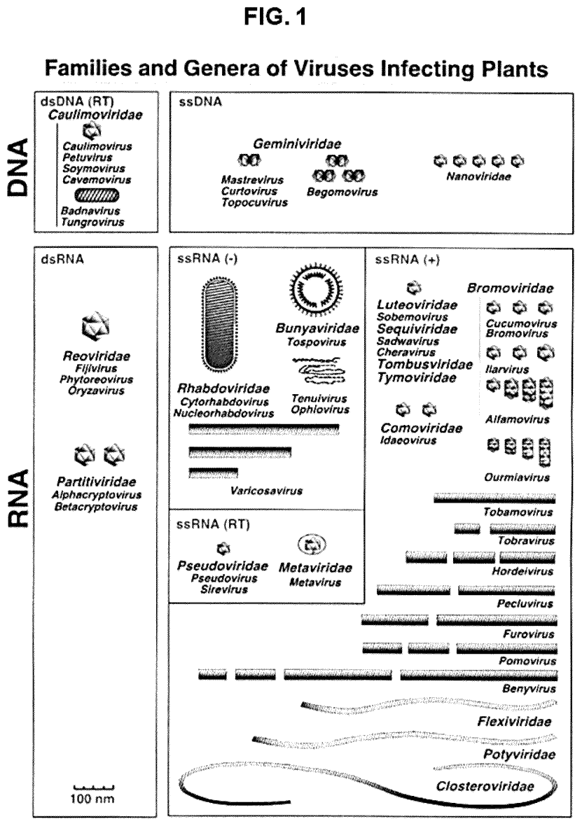

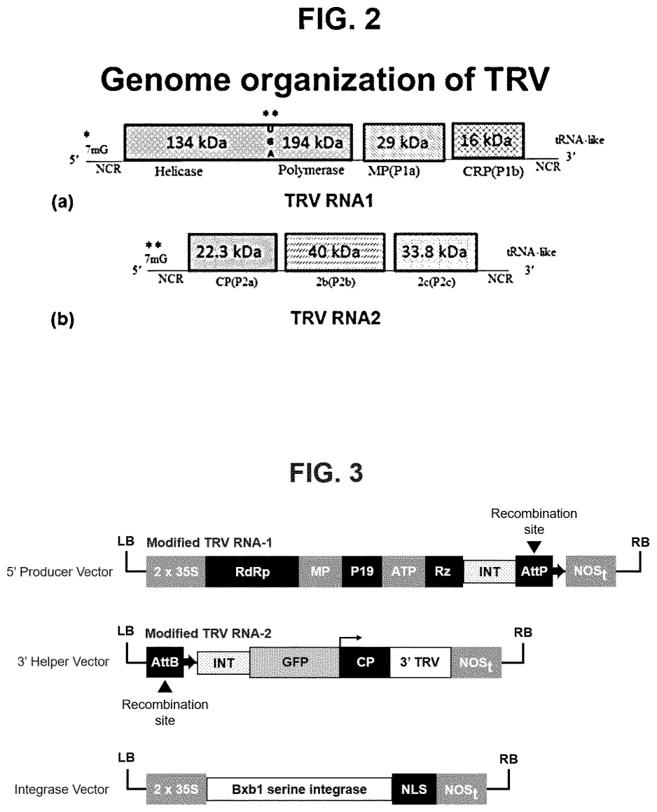

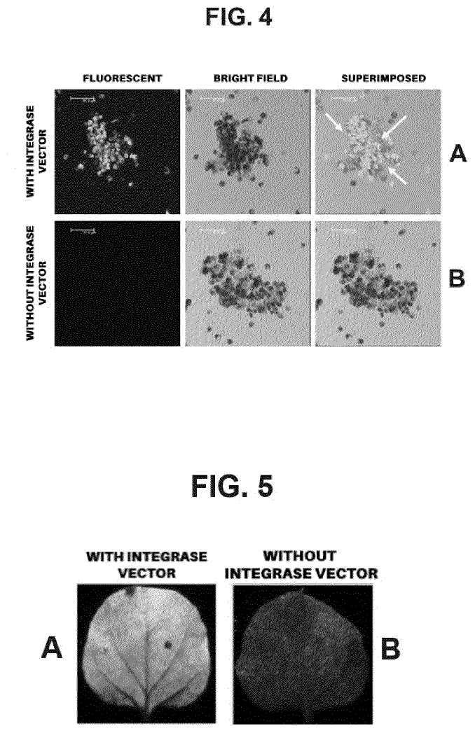

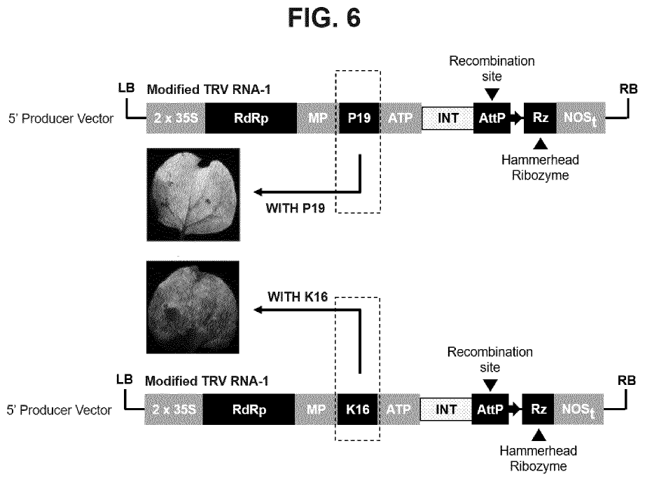

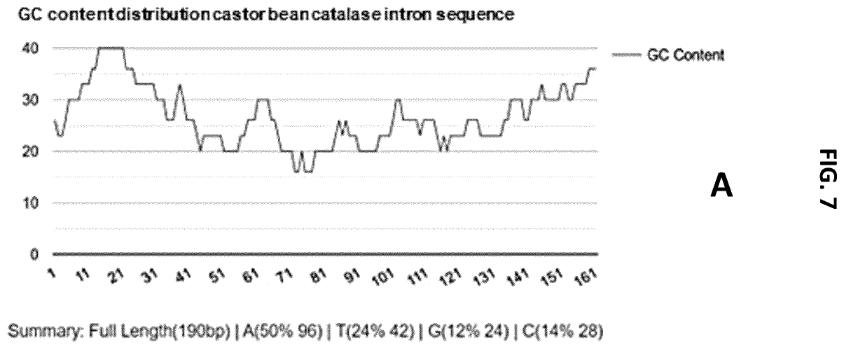

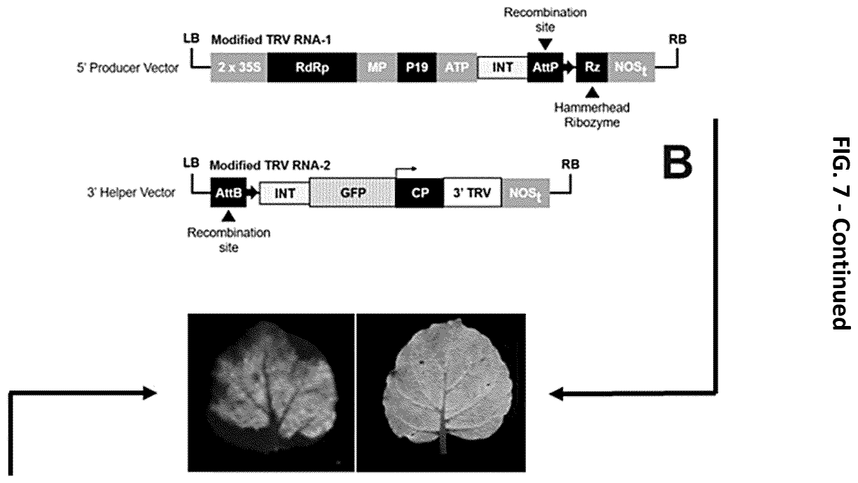

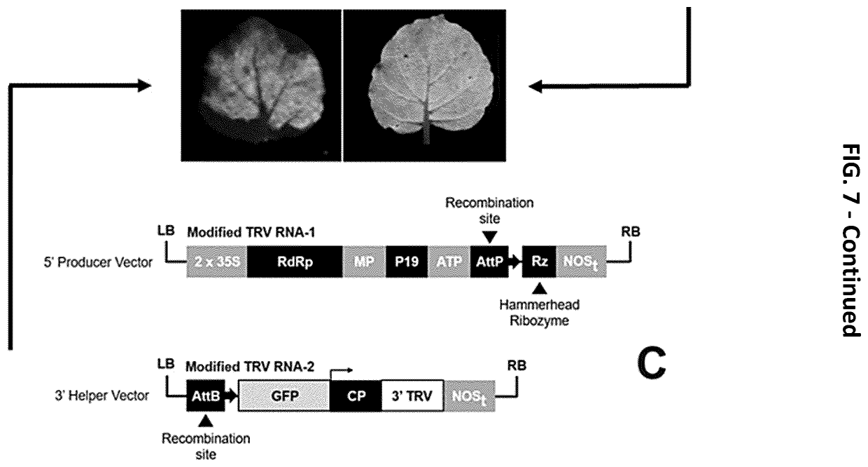

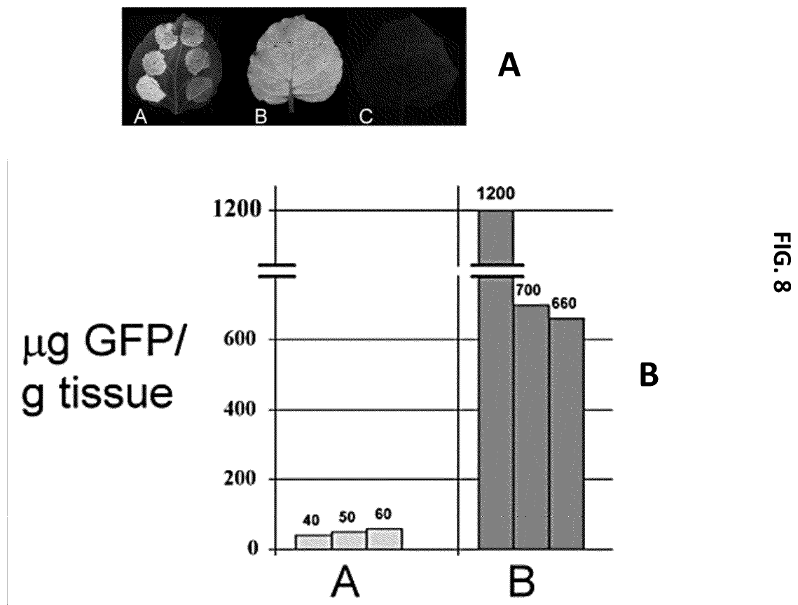

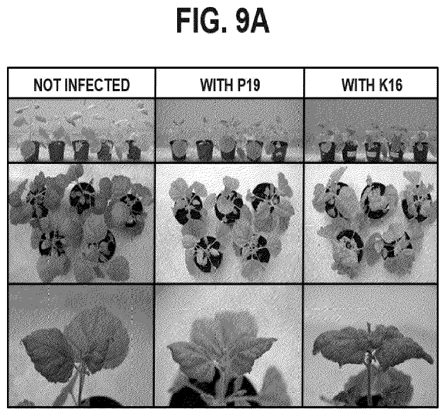

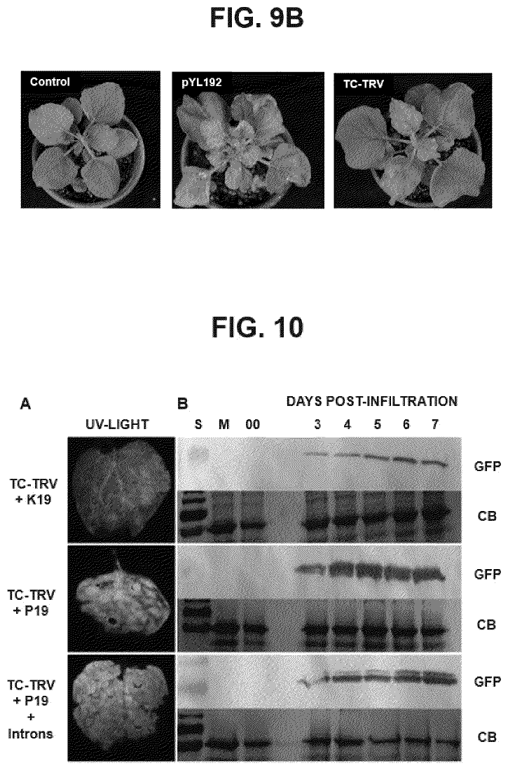

1. A method for the high-level heterologous expression of a polynucleotide of interest in a plant host which comprises the following steps:
A) transforming said plant host with one or more suspension(s) of Agrobacteria, wherein said one or more suspension(s) of Agrobacteria are containing in T-DNA a heterologous DNA having a sequence encoding a trans-complementary viral replicon operably linked to a transcriptional promotor, wherein said trans-complementary viral replicon contains:
I) coding sequences that have one or more viral replicon functions of a plant RNA viral replicon, and
II) a polynucleotide of interest,
wherein said one or more viral replicon functions of a plant RNA viral replicon, derived from a plus-sense single-stranded RNA-virus comprise:
i) a producer vector enabling in trans cell-to-cell movement of the trans-complementary viral replicon;
ii) a helper vector, wherein said helper vector is i) capable of systemic movement in said plant host both in the presence of said producer vector, and ii) capable of expressing a polynucleotide of interest in said plant host by the help of one or more proteins necessary for systemic movement, either by replication, cell-to-cell and/or long distance movement, or with the help of said producer vector; and
iii) an integrase vector which enables the in planta assembly of component i) and ii), being the producer and helper vector, thereby enabling the resulting components to be recombined inside the plant cell and leading to the creation of a vector sequence within a fully functional infective replicon.
B) maintaining the plant under conditions and for a sufficient time to allow the producer vector, the helper vector, and the integrase vector to recombine inside the plant cell, and the producer vector to complement the helper vector, so that systemic infection is accomplished in the plant; and
C) maintaining the plant under conditions and for a sufficient time in order for the polynucleotide of interest to be expressed in at least some plant cells.

2. The method of claim 1, wherein the plus-sense single-stranded RNA-virus is a Tobravirus.

3. The method of claim 2, wherein the Tobravirus is a Tobacco Rattle Virus (TRV)

4. The method of claim 3, wherein the native K16 the suppressor of silencing protein of TRV RNA-1 is replaced with P19 of Tomato Bushy Stunt Virus, leading to improved RNA silencing suppression.

5. The method of claim 1 to 4, wherein the producer vector comprises a AttP recombination site, and the helper vector comprises the corresponding AttB recombination site, so that the integrase vector may provide in planta recombination of said producer and helper vector.

6. The method of claim 1 to 5, wherein the integrase vector comprises an integrase coding sequence of a serine integrase, more specifically a Bxb1 serine integrase.

7. The method of claim 1 to 6, wherein the producer vector comprise an Apoplast targeting sequence, in particular an Apoplast targeting sequence derived from Arabidobsis thaliana 2S seed storage protein 1

8. The method of claim 1 to 7, wherein both the producer and helper vector comprise recombination sites flanked with intron elements in order to improve efficiency of mRNA translation, more specifically the intron elements are of low GC content and being derived from castor bean catalase.

9. The method of claim 1 to 8, wherein the producer vector is functionalized with a unique self-cleaving hammerhead ribozyme to improve viral infectivity.

10. The method of claim 1 to 9, wherein the helper vector comprises a non-coding portion that comprises a 3′ untranslated region from TRV RNA-2.

11. The process of claim 1 to 10, wherein said suspension of Agrobacteria has a concentration of cells of said Agrobacteria corresponding to a calculated optical density at 600 nm of at most 0.001, wherein said calculated optical density is defined by an at least 1000-fold dilution of a suspension of said Agrobacteria of an OD at 600 nm of 1.0.

12. The process of claim 1 to 11, which is a process of transiently expressing said sequence of interest and comprises transient transformation of said plant host, such as a plant, plant part, or plant cell culture with a nucleic acid molecule containing said heterologous DNA.

13. The process of claim 1 to 12, wherein said transforming is done by infiltrating said plant host with said suspension of Agrobacteria.


SEQUENCE LISTING (3 sequences)

SEQ ID NO: 1 - DNA, 7795 bp, synthetic construct
  source 1..7795: mol_type = other DNA
accgtcctatataacaccacatttggccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaatggctaatggaaattttaagctttctcaacttcttaatgttgatgaaatgtctgctgaacaaagatctcatttttttgatcttatgcttactaagccagattgtgaaattggacaaatgatgcaaagagttgttgttgataaggttgatgatatgattagagaaagaaagactaaggatccagttattgttcatgaagttctttctcaaaaggaacaaaataagcttatggaaatttatccagaatttaatattgtttttaaggatgataagaatatggttcatggatttgctgctgctgaaagaaagcttcaagctcttcttcttcttgatagagttccagctcttcaagaagttgatgatattggaggacaatggtctttttgggttactagaggagaaaagagaattcattcttgttgtccaaatcttgatattagagatgatcaaagagaaatttctagacaaatttttcttactgctattggagatcaagctagatctggaaagagacaaatgtctgaaaatgaactttggatgtatgatcaatttagaaagaatattgctgctccaaatgctgttagatgtaataatacttatcaaggatgtacttgtagaggattttctgatggaaagaagaagggagctcaatatgctattgctcttcattctctttatgattttaagcttaaggatcttatggctactatggttgaaaagaagactaaggttgttcatgctgctatgctttttgctccagaatctatgcttgttgatgaaggaccacttccatctgttgatggatattatatgaagaagaatggaaagatttattttggatttgaaaaggatccatctttttcttatattcatgattgggaagaatataagaagtatcttcttggaaagccagtttcttatcaaggaaatgttttttattttgaaccatggcaagttagaggagatactatgcttttttctatttatagaattgctggagttccaagaagatctctttcttctcaagaatattatagaagaatttatatttctagatgggaaaatatggttgttgttccaatttttgatcttgttgaatctactagagaacttgttaagaaggatctttttgttgaaaagcaatttatggataagtgtcttgattatattgctagactttctgatcaacaacttactatttctaatgttaagtcttatctttcttctaataattgggttctttttattaatggagctgctgttaagaataagcaatctgttgattctagagatcttcaacttcttgctcaaactcttcttgttaaggaacaagttgctagaccagttatgagagaacttagagaagctattcttactgaaactaagccaattacttctcttactgatgttcttggacttatttctagaaagctttggaagcaatttgctaataagattgctgttggaggatttgttggaatggttggaactcttattggattttatccaaagaaggttcttacttgggctaaggatactccaaatggaccagaactttgttatgaaaattctcataagactaaggttattgtttttctttctgttgtttatgctattggaggaattactcttatgagaagagatattagagatggacttgttaagaagctttgtgatatgtttgatattaagagaggagctcatgttcttgatgttgaaaatccatgtagatattatgaaattaatgattttttttcttctctttattctgcttctgaatctggagaaactgttcttccagatctttctgaagttaaggctaagtctgataagcttcttcaacaaaagaaggaaattgctgatgaatttctttctgctaagttttctaattattctggatcttctgttagaacttctccaccatctgttgttggatcttctagatctggacttggacttcttcttgaagattctaatgttcttactcaagctagagttggagtttctagaaaggttgatgatgaagaaattatggaacaatttctttctggacttattgatactgaagctgaaattgatgaagttgtttctgctttttctgctgaatgtgaaagaggagaaacttctggaactaaggttctttgtaagccacttactccaccaggatttgaaaatgttcttccagctgttaagccacttgtttctaagggaaagactgttaagagagttgattattttcaagttatgggaggagaaagacttccaaagagaccagttgtttctggagataattctgttgatgctagaagagaatttctttattatcttgatgctgaaagagttgctcaaaatgatgaaattatgtctctttatagagattattctagaggagttattagaactggaggacaaaattatccacatggacttggagtttgggatgttgaaatgaagaattggtgtattagaccagttgttactgaacatgcttatgtttttcaaccagataagagaatggatgattggtctggatatcttgaagttgctgtttgggaaagaggaatgcttgttaatgattttgctgttgaaagaatgtctgattatgttattgtttgtgatcaaacttatctttgtaataatagacttattcttgataatctttctgctcttgatcttggaccagttaattgttcttttgaacttgttgatggagttccaggatgtggaaagtctactatgattgttaattctgctaatccatgtgttgatgttgttctttctactggaagagctgctactgatgatcttattgaaagatttgcttctaagggatttccatgtaagcttaagagaagagttaagactgttgattcttttcttatgcattgtgttgatggatctcttactggagatgttcttcattttgatgaagctcttatggctcatgctggaatggtttatttttgtgctcaaattgctggagctaagagatgtatttgtcaaggagatcaaaatcaaatttcttttaagccaagagtttctcaagttgatcttagattttcttctcttgttggaaagtttgatattgttactgaaaagagagaaacttatagatctccagctgatgttgctgctgttcttaataagtattatactggagatgttagaactcataatgctactgctaattctatgactgttagaaagattgtttctaaggaacaagtttctcttaagccaggagctcaatatattacttttcttcaatctgaaaagaaggaacttgttaatcttcttgctcttagaaaggttgctgctaaggtttctactgttcatgaatctcaaggagaaacttttaaggatgttgttcttgttagaactaagccaactgatgattctattgctagaggaagagaatatcttattgttgctctttctagacatactcaatctcttgtttatgaaactgttaaggaagatgatgtttctaaggaaattagagaatctgctgctcttactaaggctgctcttgctagattttttgttactgaaactgttcttnnnagatttagatctagatttgatgtttttagacatcatgaaggaccatgtgctgttccagattctggaactattactgatcttgaaatgtggtatgatgctctttttccaggaaattctcttagagattcttctcttgatggatatcttgttgctactactgattgtaatcttagacttgataatgttactattaagtctggaaattggaaggataagtttgctgaaaaggaaacttttcttaagccagttattagaactgctatgccagataagagaaagactactcaacttgaatctcttcttgctcttcaaaagagaaatcaagctgctccagatcttcaagaaaatgttcatgctactgttcttattgaagaaactatgaagaagcttaagtctgttgtttatgatgttggaaagattagagctgatccaattgttaatagagctcaaatggaaagatggtggagaaatcaatctactgctgttcaagctaaggttgttgctgatgttagagaacttcatgaaattgattattcttcttatatgtatatgattaagtctgatgttaagccaaagactgatcttactccacaatttgaatattctgctcttcaaactgttgtttatcatgaaaagcttattaattctctttttggaccaatttttaaggaaattaatgaaagaaagcttgatgctatgcaaccacattttgtttttaatactagaatgacttcttctgatcttaatgatagagttaagtttcttaatactgaagctgcttatgattttgttgaaattgatatgtctaagtttgataagtctgctaatagatttcatcttcaacttcaacttgaaatttatagactttttggacttgatgaatgggctgcttttctttgggaagtttctcatactcaaactactgttagagatattcaaaatggaatgatggctcatatttggtatcaacaaaagtctggagatgctgatacttataatgctaattctgatagaactctttgtgctcttctttctgaacttccacttgaaaaggctgttatggttacttatggaggagatgattctcttattgcttttccaagaggaactcaatttgttgatccatgtccaaagcttgctactaagtggaattttgaatgtaagatttttaagtatgatgttccaatgttttgtggaaagtttcttcttaagacttcttcttgttatgaatttgttccagatccagttaaggttcttactaagcttggaaagaagtctattaaggatgttcaacatcttgctgaaatttatatttctcttaatgattctaatagagctcttggaaattatatggttgtttctaagctttctgaatctgtttctgatagatatctttataagggagattctgttcatgctctttgtgctctttggaagcatattaagtcttttactgctctttgtactctttttagagatgaaaatgataaggaacttaatccagctaaggttgattggaagaaggctcaaagagctgtttctaatttttatgattggatggaagataagtctcttgttactcttaagaagaagacttttgaagtttctaagttttctaatcttggagctattgaactttttgttgatggaagaagaaagagaccaaagtattttcatagaagaagagaaactgttcttaatcatgttggaggaaagaagtctgaacataagcttgatgtttttgatcaaagagattataagatgattaagtcttatgcttttcttaagattgttggagttcaacttgttgttacttctcatcttccagctgatactccaggatttattcaaattgatcttcttgattctagacttactgaaaagagaaagaagggaaagactattcaaagatttaaggctagagcttgtgataattgttctgttgctcaatataaggttgaatattctatttctactcaagaaaatgttcttgatgtttggaaggttggatgtatttctgaaggagttccagtttgtgatggaacttatccattttctattgaagtttctcttatttgggttgctactgattctactagaagacttaatgttgaagaacttaattcttctgattatattgaaggagattttactgatcaagaagtttttggagaatttatgtctcttaagcaagttgaaatgaagactattgaagctaagtatgatggaccatatagaccagctactactagaccaaagtctcttctttcttctgaagatgttaagagagcttctaataagaagaattcttctatggaaagagctattcaaggaaatgatgctagagaacaagctaattctgaaagatgggatggaggatctggaggaactacttctccatttaagcttccagatgaatctccatcttggactgaatggagacttcataatgatgaaactaattctaatcaagataatccacttggatttaaggaatcttggggatttggaaaggttgtttttaagagatatcttagatatgatagaactgaagcttctcttcatagagttcttggatcttggactggagattctgttaattatgctgcttctagattttttggatttgatcaaattggatgtacttattctattagatttagaggagtttctattactgtttctggaggatctagaactcttcaacatctttgtgaaatggctattagatctaagcaagaacttcttcaacttgctccaattgaagttgaatctaatgtttctagaggatgtccagaaggaactgaaacttttgaaaaggaatctgaaatggctaataagctttttcttgtttgtgctgctcttgctctttgttttcttcttactaatgctcatttaaagatcaaaaagaggaagtaaaagaaccaatcctgggaaaagagaaaaataaaaaaactcgaaactagaaagaaatttgactagataaaaaattaactaaccaataccacatttataatgtatcgaaattgactattagactaatgaaataaagcacacagatactactactactatcaatgtcggtttgtctggtcaaccaccgcggtctcagtggtgtacggtacaaaccggcatgctgatgagtccgtgaggacgaaacgagtaagctcgtcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgactgtcctatataaccgcccatttg

SEQ ID NO: 2 - DNA, 2581 bp, synthetic construct
  source 1..2581: mol_type = other DNA
accgtcctatataacaccacatttgggcttgtcgacgacggcggtctccgtcgtcaggatcatcatttaaagatcaaaaagaggaagtaaaagaaccaatcctgggaaaagagaaaaataaaaaaactcgaaactagaaagaaatttgactagataaaaaattaactaaccaataccacatttataatgtatcgaaattgactattagactaatgaaataaagcacacagatactactactactatcaatgtcaaggatgaacttatgtctaagggagaagaactttttactggagttgttccaattcttgttgaacttgatggagatgttaatggacataagttttctgtttctggagaaggagaaggagatgctacttatggaaagcttactcttaagtttatttgtactactggaaagcttccagttccatggccaactcttgttactactttttcttatggagttcaatgtttttctagatatccagatcatatgaagcaacatgatttttttaagtctgctatgccagaaggatatgttcaagaaagaactattttttttaaggatgatggaaattataagactagagctgaagttaagtttgaaggagatactcttgttaatagaattgaacttaagggaattgattttaaggaagatggaaatattcttggacataagcttgaatataattataattctcataatgtttatattatggctgataagcaaaagaatggaattaaggttaattttaagattagacataatattgaagatggatctgttcaacttgctgatcattatcaacaaaatactccaattggagatggaccagttcttcttccagataatcattatctttctactcaatctgctctttctaaggatccaaatgaaaagagagatcatatggttcttcttgaatttgttactgctgctggaattactcatggaatggatgaactttataagatggctggatcttatggagaaacttttgatggaaagattcttgatgatctttctggagcttgggttgaaaagcataattggtctgatattcttagaagacttactaagattaagtttgctcttcaagctgatagagatatgattccaggaattgttgaagatctttctactgaaattccagttgatgaaaatactagatttccatctggaaaggtttatcatcttcttactaaggaaatgcttatggctattgaagctattcatgctgcttcttcttttaagagaagagctgaagaaaagaataatgttaatccaagacaaagatttgaagctgaatcttcttcttctcaacttccatctggaggacttgttgttagaccagctgctggatctggagattcttctcttggagaagatcttttttctaattctaagcttgatgatgcttctactgcttttcataagtctcttgctactcttaagggatctagaccaaaggctgttgttcaaagaacttttgaaaaggaatattctcttagatggactgctgctgctccagttgctgctgctggaggaactccaccaggaggaagatcttggacttggaatcttgttttccgggaccatcttgatatttacaacttaacttgttctccccctttcctggtgagcagtgcggttgtagatggacactacgcacgggataagttcgttagttttcaaggtgtatgtgggttcaatccgatgtttccagacgtgaatggtttgaagagtagttggtctttaggtcgtcaactggatgatatacggtctcaaaagaaagaggtttctggaacaaaccaggagccaaactattactacgatggcgacacactaaaaccgatcggttcaggtgcgagcgttgtaggagagagaaggccgggatggaggtgaatgtcccgaagacattaaactacgttctttaagtagatccgtgcctgaagttttaggttcaatttaaacctacgagattgacattctcgactgatcttgattgatcggtaagtcttttgtaatttaattttctttttgattttattttaaattgttatctgtttctgtgtatagactgtttgagatcggcgtttaggctcattgtcttaccataggggaacggactttgtttgtgttgttattttatttgtattttattaaaattctcaacgatctgaaaaagcctcgcgctaagagattgttggggggtgagtaagtacttttaaagtgatgatggttacaaaggcaaaaggggtaaaacccctcgcctacgtaagcgttattacgcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgactgtcctatataaccgcccatttg

SEQ ID NO: 3 - DNA, 2603 bp, synthetic construct
  source 1..2603: mol_type = other DNA
accgtcctatataacaccacatttggccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaatgagagctcttgttgttattagactttctagagttactgatgctactacttctccagaaagacaacttgaatcttgtcaacaactttgtgctcaaagaggatgggatgttgttggagttgctgaagatcttgatgtttctggagctgttgatccatttgatagaaagagaagaccaaatcttgctagatggcttgcttttgaagaacaaccatttgatgttattgttgcttatagagttgatagacttactagatctattagacatcttcaacaacttgttcattgggctgaagatcataagaagcttgttgtttctgctactgaagctcattttgatactactactccatttgctgctgttgttattgctcttatgggaactgttgctcaaatggaacttgaagctattaaggaaagaaatagatctgctgctcattttaatattagagctggaaagtatagaggatctcttccaccatggggatatcttccaactagagttgatggagaatggagacttgttccagatccagttcaaagagaaagaattcttgaagtttatcatagagttgttgataatcatgaaccacttcatcttgttgctcatgatcttaatagaagaggagttctttctccaaaggattattttgctcaacttcaaggaagagaaccacaaggaagagaatggtctgctactgctcttaagagatctatgatttctgaagctatgcttggatatgctactcttaatggaaagactgttagagatgatgatggagctccacttgttagagctgaaccaattcttactagagaacaacttgaagctcttagagctgaacttgttaagacttctagagctaagccagctgtttctactccatctcttcttcttagagttcttttttgtgctgtttgtggagaaccagcttataagtttgctggaggaggaagaaagcatccaagatatagatgtagatctatgggatttccaaagcattgtggaaatggaactgttgctatggctgaatgggatgctttttgtgaagaacaagttcttgatcttcttggagatgctgaaagacttgaaaaggtttgggttgctggatctgattctgctgttgaacttgctgaagttaatgctgaacttgttgatcttacttctcttattggatctccagcttatagagctggatctccacaaagagaagctcttgatgctagaattgctgctcttgctgctagacaagaagaacttgaaggacttgaagctagaccatctggatgggaatggagagaaactggacaaagatttggagattggtggagagaacaagatactgctgctaagaatacttggcttagatctatgaatgttagacttacttttgatgttagaggaggacttactagaactattgattttggagatcttcaagaatatgaacaacatcttagacttggatctgttgttgaaagacttcatactggaatgtctagaagatggaaggaagaacttgatgaagaacttcaaagaaagagagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgactgtcctatataaccgcccatttg